SEQUENCE LISTING (6 sequences)

SEQ ID NO: 1 - DNA, 23 bp, Artificial Sequence
  Guide RNA overlapping the PLP1 point mutation (c.254T>G)
ccagcaggcgggccccataaagg

SEQ ID NO: 2 - DNA, 155 bp, Artificial Sequence
  Sense strand of genomic sequence overlapping the PLP1 point mutation (c.254T>G)
ctacctgttaatgcaggatccatgccttccagtatgtcatctatggaactgcctctttcttcttcctttatggggccctcctgctggctgagggcttctacaccaccggcgcagtcaggcagatctttggcgactacaagaccaccatctgcggc

SEQ ID NO: 3 - DNA, 155 bp, Artificial Sequence
  Anti-sense strand of genomic sequence overlapping the PLP1 point mutation (c.254T>G)
gccgcagatggtggtcttgtagtcgccaaagatctgcctgactgcgccggtggtgtagaagccctcagccagcaggagggccccataaaggaagaagaaagaggcagttccatagatgacatactggaaggcatggatcctgcattaacaggtag

SEQ ID NO: 4 - protein, 46 aa, Artificial Sequence
  Partial wild-type amino acid sequence of PLP1 overlapping the PLP1 point mutation (c.254T>G)
IHAFQYVIYGTASFFFLYGALLLAEGFYTTGAVRQIFGDYKTTICG

SEQ ID NO: 5 - DNA, 25 bp, Artificial Sequence
  Partial mutant parental sense strand nucleotide sequence for PLP1c.254G
tttatggggcccgcctgctggctga

SEQ ID NO: 6 - DNA, 25 bp, Artificial Sequence
  Partial corrected sense strand nucleotide sequence for PLP1c.254T
tttatggggccctcctgctggctga

(19) United States
(12) Patent Application Publication
(10) Pub. No.: US 2021/0163888 A1
(43) Pub. Date: Jun. 3, 2021

(54) INDUCTION OF MYELINATING OLIGODENDROCYTES IN HUMAN CORTICAL SPHEROIDS

(71) Applicant: Case Western Reserve University, Cleveland, OH (US)

(72) Inventors: Paul Tesar, Wickliffe, OH (US); Mayur Madhavan, Beachwood, OH (US); Zachary Nevin, Cleveland Heights, OH (US)

(21) Appl. No.: 17/048,289

(22) PCT Filed: Apr. 16, 2019
(86) PCT No.: PCT/US2019/027685

§ 371 Date: Oct. 16, 2020

Related U.S. Application Data
(60) Provisional application No. 62/700,472, filed on Jul. 19, 2018.
(60) Provisional application No. 62/658,901, filed on Apr. 17, 2018.

(51) Int. Cl. C12N 5/079; G01N 33/50

(52) U.S. Cl. CPC C12N 5/0622; G01N 33/5058; C12N 2501/105; C12N 2501/135; C12N 2513/00; C12N 2503/02; C12N 2501/999; C12N 2506/45; C12N 2501/395

(57) ABSTRACT

The invention described herein provides a method for generating oligocortical spheroids from (human) pluripotent stem cells. The cortical spheroids so generated produces maturing oligodendrocytes that can, for example, myelinate axons, and model myelin disease and drug effects.

### REFERENCE TO RELATED APPLICATIONS

This international patent application claims the benefit of the filing date to U.S. Provisional Patent Application Nos. 62/658,901, filed on Apr. 17, 2018, and 62/700,472, filed on Jul. 19, 2018, the entire contents of each of the above applications, including any drawings and sequence listings, are hereby incorporated herein by reference.

### GOVERNMENT SUPPORT

This invention was made with government support under the grant(s) NS093357, NS095280, GM007250, HD084167, and CA043703 awarded by the National Institutes of Health. The government has certain rights in the invention.

### BACKGROUND OF THE INVENTION

Human corticogenesis is a complex process that requires the coordinated generation, migration, and maturation of distinct cell populations. While many groups have generated oligodendrocytes through in vitro 2D cultures and forced aggregation of differentiating neural cells, hPSC-derived cortical spheroids harness intrinsic differentiation programs to recapitulate regional organization and cortical layering present in the developing human brain.

Advances in the generation of 3-dimensional (3D) tissues in vitro are improving the ability to study human neurodevelopment and disease. Human pluripotent stem cell (hPSC)-derived 3D cultures—called “organoids” or “spheroids”—recapitulate complex developmental processes, cell-cell interactions, microenvironments, tissue architectures, and extended temporal dynamics that are inaccessible in traditional in vitro cultures.

Multiple groups have developed protocols to model the coordinated rounds of cell proliferation, migration, organization, and maturation required to pattern the human cerebral cortex. These pluripotent stem cell-derived “cortical spheroids” have been shown to generate multiple cortical cell types—including neural progenitors, mature neuron subtypes, and astrocytes—which self-organize into distinct cortical layers, and establish functional neural networks. However, while single-cell analyses of cortical spheroids have identified transcriptional profiles suggesting the presence of oligodendrocyte progenitor cells (OPCs), and rare oligodendrocytes have been identified in isolation, no protocol has yet demonstrated reproducible generation and maturation of oligodendrocytes, the myelinating glia of the central nervous system (CNS) and the third major cell type of neural origin.

### SUMMARY OF THE INVENTION

In one aspect, the invention provides a method for generating an oligocortical spheroid (OCS) from pluripotent stem cells (PSCs), the method comprising: a) generating a neurocortical spheroid (NCS) through neurocortical patterning of said pluripotent stem cells; b) subjecting said neurocortical spheroid to timed exposure to defined oligodendrocyte lineage growth factors and/or hormones, to promote proliferation, survival and/or expansion of native oligodendrocyte progenitor cell (OPC) populations within said neurocortical spheroid, thereby generating the oligocortical spheroid; wherein said oligocortical spheroid contain oligodendrocyte progenitor cells (OPCs) capable of differentiating into myelinating oligodendrocytes (ODCs) that are capable of myelinating axons.

In certain embodiments, the defined oligodendrocyte lineage growth factors and hormones include platelet-derived growth factor (PDGF), such as PDGF-AA (PDGF-AA), and insulin-like growth factor-1 (IGF-1).

In certain embodiments, the defined oligodendrocyte lineage growth factors and hormones include PDGF-AA, PDGF-AB, FGF-2, VEGF, or a combination thereof; and insulin or IGF-1 or a combination thereof.

In certain embodiments, the method further comprises timed exposure to additional growth factors and/or hormones to induce oligodendrocyte differentiation.

In certain embodiments, the additional growth factors and/or hormones comprise thyroid hormone (T3), clemastine, and/or ketoconazole.

In certain embodiments, step b) is carried out at a time equivalent to about 10 weeks post conception, or about 50-60 days after the beginning of step a).

In certain embodiments, the timed exposure to additional growth factors and/or hormones to induce oligodendrocyte differentiation is carried out at a time equivalent to about 14 weeks post conception, or about 60-70 days after the beginning of step a).

In certain embodiments, the pluripotent stem cells are from a human embryonic stem cell line, or from an induced pluripotent stem cell (iPSC) line.

In certain embodiments, step b) is carried out over a period of about 6, 7, 8, 9, 10, 11, 12, 13, 14, or 15 days.

In certain embodiments, the neurocortical spheroids at the end of step a) contain substantially no oligodendrocyte lineage cells. The lack of oligodendrocyte lineage cells can be verified by any markers of the oligodendrocyte lineage cells, such as one or more canonical OPC markers, e.g., transcription factors OLIG2 and SOX10.

In certain embodiments, the oligocortical spheroid at the end of step b) contains substantially increased OPCs compared to age-matched neurocortical spheroids untreated by step b). The increased OPCs can be detected and/or quantitated by, for example, increased immunostaining of one or more canonical OPC markers. Suitable OPC markers may include: transcription factor specific for OPC, such as OLIG2 and SOX10, oligodendrocyte membrane protein marker, such as proteolipid protein 1 (PLP1), and transcription factor specifically expressed in oligodendrocytes in the CNS. such as MYRF.

In certain embodiments, the pluripotent stem cells are iPSC isolated from a subject having a disease. According to this embodiment, OCS produced from iPSC isolated from diseased individual can be a valuable model for treating the disease.

In certain embodiments, the disease is characterized by a defect in myelin production, or a defect caused by/associated with loss of myelin or loss of myelin function.

In certain embodiments, the disease is Pelizaeus-Merzbacher disease (PMD). For example, the PMD may be characterized by a deletion of the entire PLP1 locus, a duplication of the entire PLP1 locus, or a point mutation in PLP1 (such as c.254T>G).

Another aspect of the invention provides an oligocortical spheroid generated using any of the methods of the invention.

Another aspect of the invention provides an oligocortical spheroid developed from pluripotent stem cells, wherein the oligocortical spheroid contains oligodendrocyte progenitor cells (OPCs) capable of differentiating into myelinating oligodendrocytes that are capable of myelinating axons.

In certain embodiments, the oligocortical spheroid further comprises myelinating oligodendrocytes that are capable of myelinating axons.

Another aspect of the invention provides a method for screening for a drug effective to treat a disease characterized by a defect in myelin production, or a defect caused by /associated with loss of myelin or loss of myelin function, the method comprising contacting a plurality of candidate drugs from a library of candidate drugs, each individually with an oligocortical spheroid developed from pluripotent stem cells from an individual having said disease, and identifying one or more candidate drugs that alleviate the defect in myelin production, restore myelin amount and/or function, or prevent myelin loss as being effective to treat said disease.

In certain embodiments, the method further comprises administering the candidate drug identified as being effective to an animal having the disease. For example, the individual having the disease may be a human, and the animal can be a mouse as a model for the disease.

It should be understood that any one embodiment described herein can be combined with one or more other embodiments, including those embodiments described only in the examples or claims, unless expressly disclaimed or improper.

### BRIEF DESCRIPTION OF THE DRAWINGS

FIGS. 1A-1F show generation of oligodendrocytes in human cortical spheroids. FIG. 1A is a schematic of spheroid generation. Protocols to generate neurocortical spheroids (NCS) and oligocortical spheroids (OCS) were the same until week 8, after which time neurocortical spheroids were grown in basal media, while oligocortical spheroids were treated with PDGF-AA/IGF-1 from day 50-60 and T3 from day 60-70. Differentiation of oligodendrocytes was assessed at week 14. Colors depict neurons (magenta), astrocytes (red) and OPCs/Oligodendrocytes (green). FIGS. 1B and 1C are representative fluorescence images of week 14, H7 spheroids generated with the (a) neurocortical protocol or (b) oligocortical protocol. For each, similar results were obtained from 3 independent batches of spheroids generated from 4 separate lines. Scale bar, 50 μm. FIG. 1B shows that neurocorticol protocol spheroids generate neurons (Neurofilament:magenta) and astrocytes (GFAP:red), but no oligodendrocytes (PLP1:green). FIG. 1C shows that oligocortical protocol spheroids generate neurons (Neurofilament:magenta), astrocytes (GFAP:red), and oligodendrocytes (PLP1:green). Inset, oligodendrocyte morphology at higher magnification.

FIG. 1D shows quantification of MYRF, a nuclear marker of the oligodendrocyte lineage, in week 14 spheroids generated with the neurocortical or oligocortical protocols as well as with either PDGF-AA and IGF-1 or T3 only. MYRF-positive cells were counted from four planes from four or five individual spheroids (n=4, PDGF/IGF or T3 treatments, n=5, NCS and OCS) for each treatment condition from lines H7, H9 and CWRU191 cell line and averaged (white boxes). Error bars, s.d. n=3 spheroids from same batch were used for externally validated line RUES1. FIG. 1E shows neuron, astrocyte, and oligodendrocyte gene expression in neurocortical and oligocortical spheroids. Heat maps consist of 100 most cell-specific transcripts for each cell type. Oligodendrocyte- as well as astrocyte-specific genes are upregulated in oligocortical compared to neurocortical spheroids. FIG. 1F shows that neuron-, astrocyte-, and oligodendrocyte-specific gene expression from data in FIG. 1E. Box spans first and third quartiles, split by the mean; whiskers extend to maximum and minimum values. FIGS. 1E and 1F show RNA-seq from 5 spheroids for each condition. Paired non-parametric Wilcoxon matched pairs signed-rank test was used to determine significance.

FIGS. 2A-2L show maturation of oligodendrocytes in oligocortical spheroids. FIG. 2A is a schematic of oligocortical spheroid generation. Colors as in FIG. 1A. FIGS. 2B-2D are representative fluorescence images of week 20, H7 oligocortical spheroids. Similar results were obtained from 2 independent batches of spheroids. Scale bar, 50 μm. FIG. 2B shows robust generation of oligodendrocyte lineage (MYRF:magenta), CTIP2-positive (yellow) early-born neurons and SATB2-positive (cyan) late-born neurons. FIG. 2C shows linear process formation in maturing oligodendrocytes (PLP1:green). FIG. 2D is immunostaining for MBP (red), a marker of mature myelin, showing punctate MBP expression indicative of an early stage of maturation. FIGS. 2E-2G are representative EM of week 20, H7 oligocortical spheroids. EM results were obtained from a single batch of 3 spheroids. Scale bar, 1 μm. FIG. 2E shows cluster of neurons undergoing myelination by oligodendrocytes. FIG. 2F shows an axon encircled by multiple layers of loosely compacted myelin. FIG. 2G shows more extensive wrapping of loosely compacted myelin encircling an axon. FIGS. 2H-2J are representative fluorescence image of week 30, H9 oligocortical spheroids. Similar results were obtained from 4 spheroids from a single batch of oligocortical spheroids. Scale bar, 50 μm. FIG. 2H shows cortical lamination and separation of CTIP2-positive (yellow) deep layers from SATB2-positive (cyan) superficial layers. MYRF-positive (magenta) oligodendrocytes are interspersed within the cortical layers. FIG. 2I shows oligodendrocyte processes (PLP1:magenta) track (arrows) neuron axons (neurofilament:yellow). FIG. 2J shows higher magnification of boxed region in FIG. 2I. FIG. 2K is electron micrograph of week 30 H9 oligocortical spheroids showing compact myelin around axons. EM results were obtained from 3 spheroids from a single batch of spheroids Scale bar, 1 μm. FIG. 2L is 3D reconstruction from block face EM sections taken along the length of an axon.

FIGS. 3A-3E show cortical patterning and organization in oligocortical spheroids. FIGS. 3A and 3B are representative fluorescence images of week 8, H7 spheroids. FIG. 3A shows that, at the end of initial neurocortical patterning, spheroids generate distinct populations of neural progenitors (SOX2:yellow and Nestin:blue) that organize into ventricular-like zones. These cells are also the only actively dividing cells as marked by Ki67 (magenta). FIG. 3B shows that a TBR2-positive (blue) outer SVZ-like zone appears adjacent to the Sox2-positive (yellow) ventricular-like area. FIG. 3C is a representative fluorescence image of H7 spheroids generated with the oligocortical protocol up through PDGF-AA/IGF-1 treatment, then administered two doses of BrdU (magenta) during week 9 (day 58 and 60) to label dividing cells. BrdU-positive cells localize to SOX2-positive ventricular zones, identifying this as a primary germinal center. FIGS. 3D and 3E are representative fluorescence images of H7 spheroids generated with either the neurocortical (FIG. 3D) or oligocortical (FIG. 3E) protocol, treated with BrdU during week 9 (Day 58 and 60), and then maintained through week 14. Only oligocortical spheroids generate oligodendrocytes (MYRF:cyan), many of which are double positive for BrdU (arrows in magnification of boxed area in FIG. 3E, shown at right). Scale bars, 50 μm.

FIGS. 4A-4G show that promyelinating drugs promote the generation of oligodendrocytes in oligocortical spheroids. FIGS. 4A-4D are representative fluorescence images of week 14, H7 spheroids treated with PDGF/IGF-1 (from day 50-60) and either (FIG. 4A) DMSO, (FIG. 4B) T3, (FIG. 4C) clemastine, or (FIG. 4D) ketoconazole (from day 60-70). Whereas DMSO produced few MYRF-positive cells, T3, clemastine, and ketoconazole produced robust MYRF signal. Four spheroids from the same batch were used for analysis. Scale bar, 50 μm. FIG. 4E shows quantification of MYRF from FIGS. 4A-4D. MYRF-positive cells were counted in n=4 individual spheroids per cell line (colored points) and averaged (white bars). Error bars, s.d. Significance determined using two-tailed unpaired t-test with Welch's correction. FIGS. 4F-4G are representative EM images of week 14, H7 spheroids. Scale bar, 500 nm. FIG. 4F shows that spheroids generated with the standard oligocortical protocol (T3) demonstrate an absence of myelin. FIG. 4G shows that spheroids generated with ketoconazole in lieu of T3 demonstrate robust production of non-compact myelin encircling multiple neuronal axons.

FIGS. 5A-5N show that oligocortical spheroids recapitulate a human myelin disease phenotype. FIGS. 5A-5L are representative fluorescence images of week 14 oligocortical spheroids. Five (FIGS. 5A and 5B) or four (FIGS. 5C-5L) spheroids from the same batch were used for analysis. Scale bar, 50 μm. FIGS. 5A and 5B show CWRU198 spheroids immunostained for (FIG. 5A) PLP1:green or (FIG. 5B) MYRF:red, revealing abundant oligodendrocytes and robust PLP1 expression. FIGS. 5C-5D show PLP1 deletion spheroids immunostained for (FIG. 5C) PLP1:green or (FIG. 5D) MYRF:red, demonstrating an expected lack of PLP1 despite abundant MYRF-positive oligodendrocytes. FIGS. 5E and 5F show PLP1 duplication oligocortical spheroids immunostained for (FIG. 5E) PLP1:green or (FIG. 5F) MYRF:red, demonstrating robust PLP1 expression despite a decrease in the abundance of MYRF-positive oligodendrocytes. FIGS. 5G and 5H show PLP1 c.254T>G spheroids immunostained for (FIG. 5G) PLP1:green or (FIG. 5H) MYRF:red, demonstrating perinuclear retention of PLP1 and a decrease in MYRF-positive oligodendrocyte abundance. FIGS. 5I and 5J show PLP1 c.254T>G oligocortical spheroids treated with GSK2656157 and immunostained for (FIG. 5I) PLP1:green or (FIG. 5J) MYRF:red, demonstrating mobilization of PLP1 into oligodendrocyte process and rescue of MYRF-positive oligodendrocyte abundance. FIGS. 5K and 5L show PLP1 CRISPR-corrected c.254TG>T oligocortical spheroids immunostained for (FIG. 5K) PLP1:green or (FIG. 5L) MYRF:red, demonstrating rescue of both PLP1 perinuclear retention and oligodendrocyte abundance. FIG. 5M shows percentage of MYRF-positive oligodendrocytes per organoid in FIGS. 5A-5L. MYRF-positive cells were counted from n=5 individual spheroids of control line CWRU198 and n=4 individual spheroids per cell line (colored points) and averaged (white boxes). Error bars, s.d. Significance determined using two-tailed unpaired t-test with Welch's correction. FIG. 5N is a representative EM of week 30, PLP1 CRISPR-corrected c.254G>T oligocortical spheroids, demonstrating compact myelin encircling an axon. Three spheroids from a single batch were used for EM analysis. Scale bar, 1 μm.

FIGS. 6A-6E show generation of oligodendrocyte precursor cells in human cortical spheroids. FIG. 6A is schematic of spheroid generation. The protocols to generate neurocortical spheroids (NCS) and oligocortical spheroids (OCS) were the same until week 8. Neurocortical spheroids were grown in basal media, while oligocortical spheroids were treated with PDGF-AA/IGF-1 to generate OPCs from day 50-60. Increase in OPC numbers was assessed at the end of week 9. Colors in the schematic simulate neurons (magenta), astrocytes (red) and OPCs/Oligodendrocytes (green). FIGS. 6B-6C are representative fluorescence images of week 8 (FIG. 6B) and week 9 (FIG. 6C) H7 spheroids generated with the neurocortical protocol. These spheroids do not generate OPCs (OLIG2:yellow and SOX10:magenta). Scale bar, 50 μm for FIGS. 6B-6D. FIG. 6D is a representative fluorescence image of week 9, H7 spheroids generated with the oligocortical protocol up through treatment with PDGF-AA/IGF-1. These spheroids generate OPCs (OLIG2:yellow and SOX10:magenta). Arrows show OLIG2/SOX10 double-positive cells. FIG. 6E shows quantification of OLIG2-positive and SOX10/OLIG2-double positive OPCs in week 9 spheroids generated with the neurocortical or oligocortical protocol. Cells were counted from three planes each from five individual spheroids (colored points) of lines H7, H9 and CWRU191 and averaged (white boxes). Error bars are standard deviation, n=5 spheroids from the same batch per line.

FIGS. 7A-7C show validation of the oligocortical protocol in three additional human pluripotent lines. FIG. 7A are representative fluorescence images of PLP1 in week 14 oligocortical spheroids generated from H9, CWRU191, and RUES1. Similar results were obtained from 3 independent batches of spheroids for H9, CWRU191 and CWRU198 and one batch of RUES1. Scale bar, 50 μm. FIG. 7B are representative fluorescence images of MYRF in week 14 oligocortical spheroids generated from H9, CWRU191, and RUES1. Similar results were obtained from 3 independent batches of spheroids for H9, CWRU191 and CWRU198 and one batch of RUES1. Scale bar, 50 μm. FIG. 7C is a schematic of MYRF quantification in FIG. 1D with representative fluorescence images of MYRF in a single week 14 oligocortical spheroid generated from H7. The four panels (1-4) demonstrate four equally magnified, equally sized, and consistently distributed areas that were imaged and counted per spheroid. The reported % MYRF-positive cells per spheroid is the average of these four images. Scale bar, 50 μm.

FIGS. 8A-8C show maturation of oligodendrocytes from additional pluripotent lines. FIG. 8A shows representative fluorescence images of MYRF and PLP1 expression in week 20, H9, CWRU191, and RUES1 oligocortical spheroids. Results are representative of spheroids generated from 2 independent batches of lines H9 and CWRU191 and 1 batch of line RUES1. Scale bar, 50 μm. FIG. 8B shows representative EM images of multiple loosely compacted myelin wraps around axons in week 20, H9 and CWRU191 oligocortical spheroids. EM analysis was performed on 3 spheroids from the same batch for each line. EM analysis of RUES1 was not performed. Scale bar, 1 μm. FIG. 8C shows representative fluorescence images of Sox10 and MYRF expression in week 14 and 20 H7 oligocortical spheroids. Results are representative of spheroids generated from 2 independent batches. Scale bar, 50 μm.

FIG. 9 is BrdU based fate mapping of oligodendrocytes in oligocortical spheroids. Representative fluorescence images of two additional H7, and two H9, and two CWRU191 spheroids generated with the oligocortical protocol up through PDGF-AA/IGF-1 treatment, then administered two doses of BrdU during week 9 (day 58 and 60) to label dividing cells, are shown. After the second BrdU pulse, a majority of BrdU-positive (magenta) cells localize with SOX2-positive (yellow) and Vimentin-positive (blue) cells. By Week 14, some of the BrdU labelled cells are double-positive (arrows in high magnification inset) for the oligodendrocyte marker MYRF (cyan). Pulse chase experiments were performed on a single batch of spheroids from each line, and 4 spheroids per line were analyzed. Scale bar, 50 μm.

FIG. 10 is single cell analysis of cell populations in week 12 oligocortical spheroids. Shown is clustering of single cell RNA-seq data from Week 12 H7 oligocortical spheroids compared to single cell human fetal brain cells generated by Nowakowski et al. 2017. A continuum of progenitor populations is evident in both data sets through visualization of progenitor markers Vimentin, SOX2, Nestin, and Sox6 while only the oligocortical spheroids show evidence of an emerging oligodendrocyte cluster (PLP1/DM20 and OMG). Single Cell RNA-seq was performed 10 spheroids from a single batch.

FIGS. 11A-11C show CRISPR correction of a PLP1 point mutation. FIG. 11A is schematic of the correction of a PLP1 point mutation (PLP1^{c.254T>G}) in patient-derived hiPSCs using a guide RNA overlapping the mutation and single strand antisense oligonucleotide donor. FIG. 11B is Sanger sequencing trace and karyotype of the mutant parental (PLP^{c.254G}) line.

FIG. 11C is Sanger sequencing trace and karyotype of the corrected (PLP1^{c.254T}) line.

### DETAILED DESCRIPTION OF THE INVENTION

Cerebral organoids provide an accessible system to examine cellular composition, interactions and organization but have lacked oligodendrocytes, the myelinating glia of the central nervous system. Described herein is a method for reproducibly generating oligodendrocytes and myelin in human pluripotent stem cell-derived “oligocortical spheroids.” Molecular features consistent with maturing oligodendrocytes appear by 20 weeks in culture, with further maturation and myelin compaction by 30 weeks.

Promyelinating drugs enhance the rate and extent of oligodendrocyte generation and myelination, while spheroids generated from patients with a genetic myelin disorder recapitulate human disease phenotypes.

Thus the subject method and the oligocortical spheroids generated thereby provide a versatile platform to study myelination of the developing central nervous system, and offer new opportunities for disease modeling and therapeutic development.

Applicant has developed a method to reproducibly induce oligodendrocyte progenitors and myelinating oligodendrocytes in cortical spheroids by exposing them to growth factors such as PDGF, IGF-1, and T3, while preserving the general organization and regional specification demonstrated in prior neuronal models. The induction of all major CNS lineages in these oligocortical spheroids provides a new opportunity to observe and perturb human cortical development and disease.

Thus in one aspect, the invention provides a method for generating an oligocortical spheroid (OCS) from pluripotent stem cells (PSCs), the method comprising: a) generating a neurocortical spheroid (NCS) through neurocortical patterning of said pluripotent stem cells; b) subjecting said neurocortical spheroid to timed exposure to defined oligodendrocyte lineage growth factors and/or hormones, to promote proliferation, survival and/or expansion of native oligodendrocyte progenitor cell (OPC) populations within said neurocortical spheroid, thereby generating the oligocortical spheroid; wherein said oligocortical spheroid contain oligodendrocyte progenitor cells (OPCs) capable of differentiating into myelinating oligodendrocytes (ODCs) that are capable of myelinating axons.

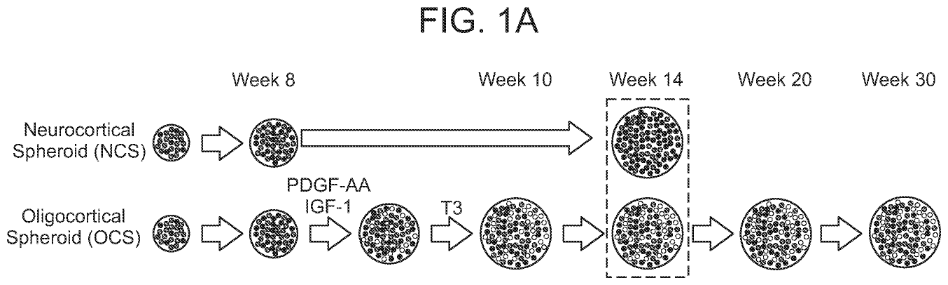

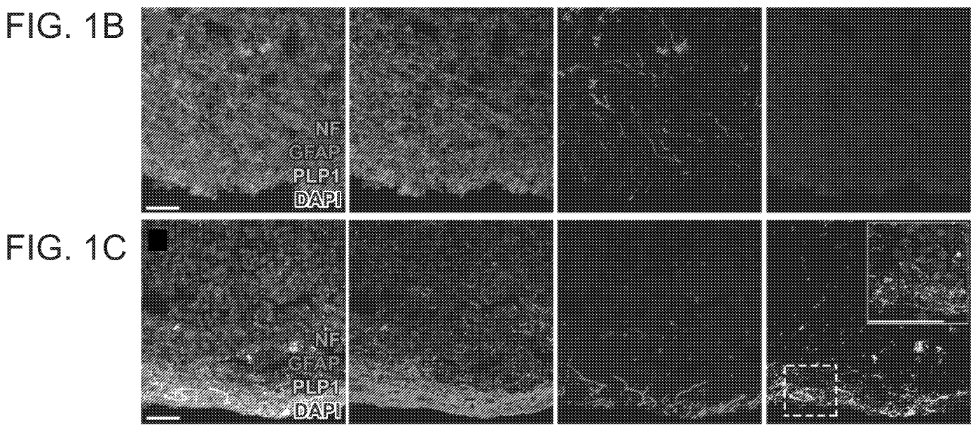

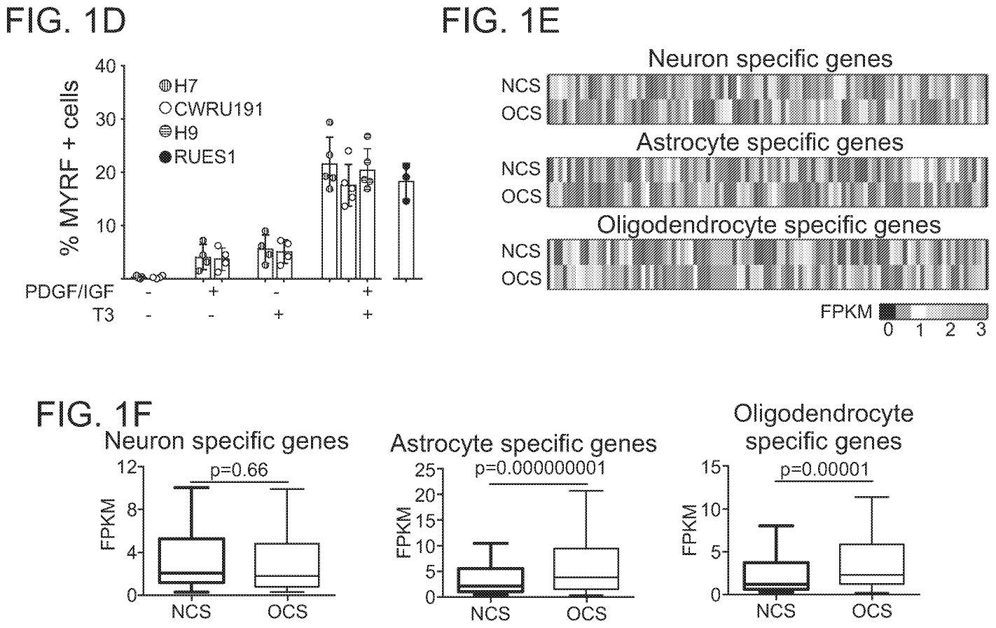

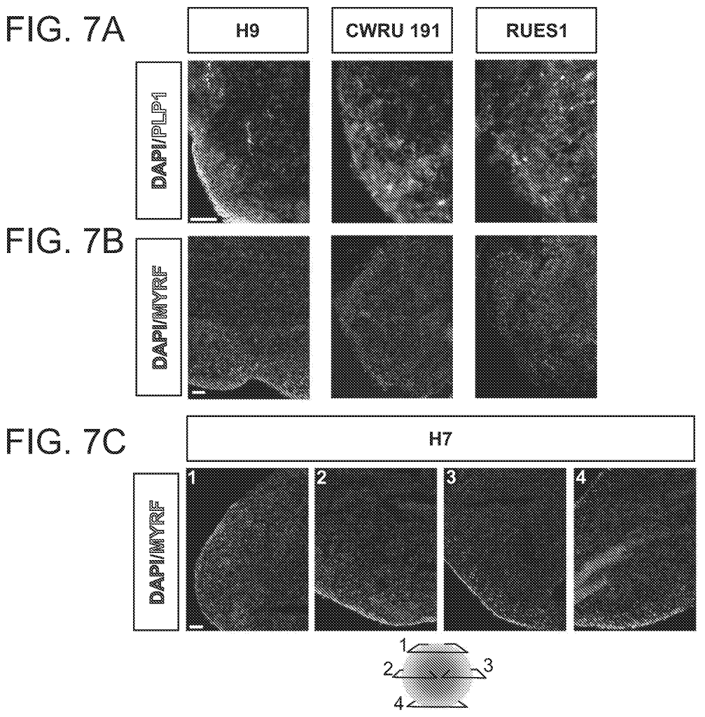

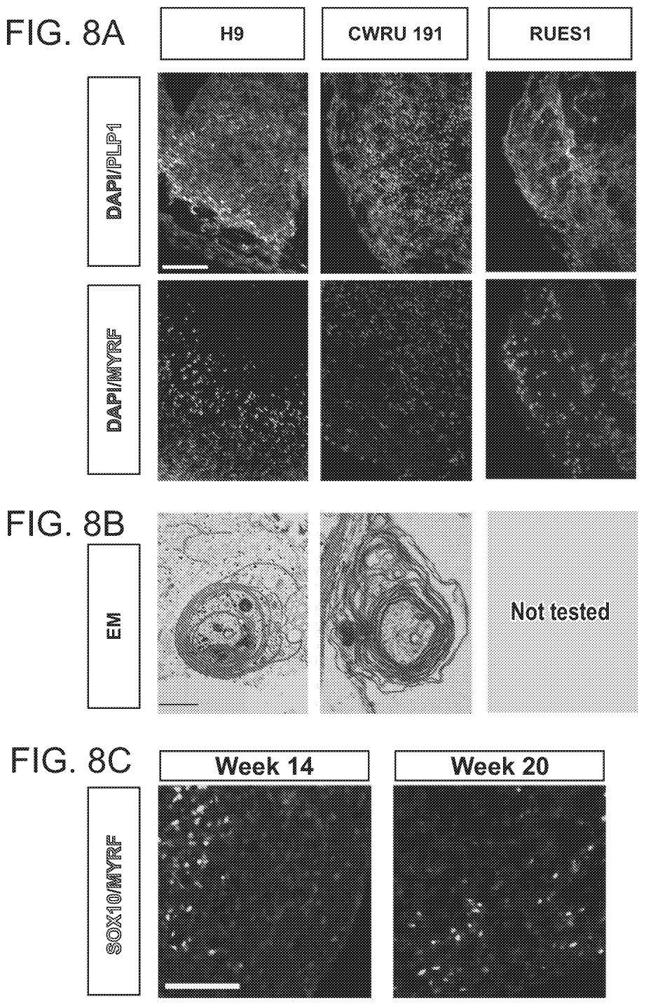

In certain embodiments, the oligocortical spheroid contain at least about 5, 6, 7, 8, 9, 10, 15, 20, 25, 30% oligodendrocyte progenitor cells (OPCs) and/or differentiated oligodendrocytes, preferably at the end of week 9, 14, or 20 after the commencement of step a). The percentage of OPCs and/or ODCs can be measured based on counting cells expressing OPC/ODC markers, such as MYRF or PLP1. The cells can be counted according to the method used for FIG. 1D or FIG. 7C (e.g., counted from four planes from four or five individual spheroids).

In certain embodiments, the defined oligodendrocyte lineage growth factors and hormones include platelet-derived growth factor (PDGF), such as PDGF-AA (PDGF-AA), and insulin-like growth factor-1 (IGF-1).

In certain embodiments, the defined oligodendrocyte lineage growth factors and hormones include PDGF-AA, PDGF-AB, FGF-2, VEGF, or a combination thereof; and insulin or IGF-1 or a combination thereof.

In certain embodiments, the method further comprises timed exposure to additional growth factors and/or hormones to induce oligodendrocyte differentiation.

Any factors known to induce oligodendrocyte differentiation from OPCs can be used in this step of the invention. In certain embodiments, the additional growth factors and/or hormones comprise thyroid hormone (T3), clemastine, and/or ketoconazole.

In certain embodiments, step b) is carried out at a time equivalent to about 10 weeks post conception, or about 50-60 days after the beginning of step a).

In certain embodiments, the timed exposure to additional growth factors and/or hormones to induce oligodendrocyte differentiation is carried out at a time equivalent to about 14 weeks post conception, or about 60-70 days after the beginning of step a).

In certain embodiments, the pluripotent stem cells are from a human embryonic stem cell line, or from an induced pluripotent stem cell (iPSC) line.

In certain embodiments, step b) is carried out over a period of about 6, 7, 8, 9, 10, 11, 12, 13, 14, or 15 days.

In certain embodiments, the neurocortical spheroids at the end of step a) contain substantially no oligodendrocyte lineage cells. The lack of oligodendrocyte lineage cells can be verified by any markers of the oligodendrocyte lineage cells. For example, the lack of oligodendrocyte lineage cells can be verified by lack of or minimal immunostaining of one or more canonical OPC markers, such as transcription factors OLIG2 and SOX10.

In certain embodiments, the oligocortical spheroid at the end of step b) contains substantially increased OPCs compared to age-matched neurocortical spheroids untreated by step b). The increased OPCs can be detected and/or quantitated by, for example, increased immunostaining of one or more canonical OPC markers. Suitable OPC markers may include: transcription factor specific for OPC, such as OLIG2 and SOX10, oligodendrocyte membrane protein marker, such as proteolipid protein 1 (PLP1), and transcription factor specifically expressed in oligodendrocytes in the CNS. such as MYRF.

In certain embodiments, the pluripotent stem cells are iPSC isolated from a subject having a disease. According to this embodiment, OCS produced from iPSC isolated from diseased individual can be a valuable model for treating the disease.

In certain embodiments, the disease is characterized by a defect in myelin production, or a defect caused by/associated with loss of myelin or loss of myelin function.

In certain embodiments, the disease is Pelizaeus-Merzbacher disease (PMD). For example, the PMD may be characterized by a deletion of the entire PLP1 locus, a duplication of the entire PLP1 locus, or a point mutation in PLP1 (such as c.254T>G).

Another aspect of the invention provides an oligocortical spheroid generated using any of the methods of the invention.

Another aspect of the invention provides an oligocortical spheroid developed from pluripotent stem cells, wherein the oligocortical spheroid contains oligodendrocyte progenitor cells (OPCs) capable of differentiating into myelinating oligodendrocytes that are capable of myelinating axons.

In certain embodiments, the oligocortical spheroid further comprises myelinating oligodendrocytes that are capable of myelinating axons.

Another aspect of the invention provides a method for screening for a drug effective to treat a disease characterized by a defect in myelin production, or a defect caused by /associated with loss of myelin or loss of myelin function, the method comprising contacting a plurality of candidate drugs from a library of candidate drugs, each individually with an oligocortical spheroid developed from pluripotent stem cells from an individual having said disease, and identifying one or more candidate drugs that alleviate the defect in myelin production, restore myelin amount and/or function, or prevent myelin loss as being effective to treat said disease.

In certain embodiments, the method further comprises administering the candidate drug identified as being effective to an animal having the disease. For example, the individual having the disease may be a human, and the animal can be a mouse as a model for the disease.

With the invention being generally described above, certain features of the invention are further described below in more detail in the sections below.

### Generation of Neurocortical Spheroids (NCS)

According to the methods of the invention, neurocortical spheroids can be generated from (human) pluripotent stem cells (hPSCs) by timed exposure to defined oligodendrocyte lineage growth factors and hormones.

An exemplary 50-day protocol is described in (Pasca et al., Functional cortical neurons and astrocytes from human pluripotent stem cells in 3D culture. Nat Methods 12, 671-678 (2015), incorporated herein by reference). Thus in one embodiment, the neurocortical spheroids are generated from (human) pluripotent stem cells (hPSCs) according to the 50-day protocol described in Pasca et al.

In another embodiment, the neurocortical spheroids are generated from (human) pluripotent stem cells (hPSCs) according to a modified version of the 50-day protocol described in Pasca et al., as briefly described herein.

Specifically, pluripotent stem cell colonies are cultured on vitronectin (e.g., Gibco #A14700). These cell colonies are harvested using an enzyme, such as dispase (e.g., Gibco #17105-041) at 37° C. for 10 minutes. Intact colonies are then transferred to individual low-adherence tissue culture surfaces (e.g., V-bottom 96-well plates from S-Bio Prime #MS-9096VZ) in a suitable volume (e.g., 200 μL) of Spheroid Starter media including a Rock inhibitor (e.g., 10 μM Y-27632 from Calbiochem #688001), an AMP-kinase inhibitor (e.g., 10 μM Dorsopmorphin from Sigma #P5499), and a TGF-β inhibitor (e.g., 10 μM SB-431542 from Sigma #S4317).

The Spheroid Starter media can be made in DMEM/F12 (Invitrogen #11320-033) containing 20% Knock out Serum (Invitrogen #12587-010), Non-essential amino acids (Invitrogen #11140050), Glutamax (Invitrogen #35050061), β-mercaptoethanol and 100 U/mL Penicillin/Streptomycin.

The same media without rock inhibitor is then used for the next five days, after which the media is changed to Neurobasal-A based spheroid media. Neurobasal-A spheroid media is Neurobasal-A medium (Invitrogen #10888022) with added B-27 serum substitute without vitamin A (Invitrogen #12587), Glutamax (Invitrogen #35050061) and 100 U/mL Penicillin/Streptomycin.

From day 7-25, 20 ng/ml FGF-2 (R&D systems #233-FB-25/CF) and 10 ng/ml EGF (R&D systems #236-EG-200) are added to the media.

Spheroids are cultured in 96-well plates through day 25, with daily half-media changes. On day 25, spheroids are transferred to ultra-low attachment tissue culture surface, such as 6-well plates from Corning #CLS3471, at a density of 8-10 spheroids per well and cultured thus through the remainder of the protocol.

Also from this point forward 1% Geltrex (Invitrogen #A15696-01) was added to the Neurobasal-A spheroid media.

Neural differentiation can be induced between days 27 and 41 by supplementing Neurobasal-A spheroid media with 20 ng/ml BDNF (R&D systems #248-BD) and 20 ng/ml NT-3 (R&D systems #267-N). Half media changes can be performed every other day between days 17 and 41.

### Generation of Oligocortical Spheroids (OCS)

To generate oligocortical spheroids, the NCS is allowed timed exposure to defined oligodendrocyte lineage growth factors and/or hormones to promote proliferation, survival and/or expansion of native oligodendrocyte progenitor cell (OPC) populations within the neurocortical spheroid.

In one embodiment, beginning on day 50, 10 ng/mL platelet-derived growth factor-AA (PDGF-AA, such as that from R&D Systems #221-AA-050) and 10 ng/mL insulin-like growth factor-1 (IGF-1, such as that from R&D Systems #291-G1-200) are added to the every-other-day media changes for 10 days to generate oligocortical spheroids.

OCS so generated contains oligodendrocyte progenitor cells (OPCs) capable of differentiating into myelinating oligodendrocytes (ODCs) that are capable of myelinating axons.

OCS so generated can be further exposed to additional growth factors and/or hormones to induce oligodendrocyte differentiation.

In one embodiment, on day 60, 40 ng/mL 3,3′,5-triiodothronine (T3, Sigma #ST2877) is added to the every-other-day media changes for 10 days. Optionally, small molecules can also be supplemented during this period. For example, 4 μM Ketoconazole and 2 μM Clemastine can be added in lieu of T3. Further, GSK2656157 may be added in addition to T3.

### Exemplary Uses

In validating the subject system, Applicant has demonstrated applications in genetic disease modeling and preclinical drug screening. The subject oligocortical spheroids could be used to study many outstanding questions, from understanding demyelination in leukodystrophies to developing remyelination strategies to treat multiple sclerosis. This system can also be utilized to explore basic questions of myelin development in different neuronal classes, myelin compaction, node and internode size modulation, and single-neuron and whole-spheroid electrophysiology.

Regional populations of oligodendrocytes arise, migrate, and mature at distinct times during embryogenesis. In mammals, ventrally derived oligodendrocytes are among the first population to arise, yet are not required for the proper myelination of the cortex and are mostly replaced by later cortex-derived oligodendrocytes. Even compared to non-human primates, the timing and duration of human myelination is regionally distinct. Human oligocortical spheroids provide an accessible system to explore these and other uniquely human aspects of myelin development.

### EXAMPLES

### Example 1 Generation of Oligocortical Spheroids

Described herein is an exemplary protocol for generating cortical spheroids derived from (human) pluripotent stem cells (hPSCs) that contain oligodendrocyte progenitor cells (OPCs) and myelinating oligodendrocytes, by timed exposure to defined oligodendrocyte lineage growth factors and hormones.

To start, Applicant generated and patterned “neurocortical spheroids” using an optimized version of a 50-day protocol (Pasca et al., Functional cortical neurons and astrocytes from human pluripotent stem cells in 3D culture. Nat Methods 12, 671-678 (2015), incorporated herein by reference). See variations in Example 7.

After initial neurocortical patterning, Applicant generated “oligocortical spheroids” by treatment with platelet-derived growth factor-AA (PDGF-AA) and insulin-like growth factor-1 (IGF-1) to drive the expansion of native OPC populations (days 50-60=“week 9”), followed by thyroid hormone (T3) to induce oligodendrocyte differentiation, and ultimately myelination (days 60-70=“week 10”) (FIG. 1A).

PDGF-AA and IGF-1 are requisite developmental mitogens that promote the proliferation and survival of OPCs, and T3 regulates and induces the generation of oligodendrocytes from OPCs in vivo. Treatment time periods were empirically determined, but mirror the initial specification of OPCs and oligodendrocytes in the human fetal brain at 10 and 14 weeks post-conception, respectively.

To assess inter-line variability and demonstrate the robustness of the protocol, Applicant initially developed the protocol using human embryonic stem cell line H7 (female). Applicant then reproduced key experiments using two additional independent hPSC lines: embryonic stem cell line H9 (female) and in-house derived induced pluripotent stem cell (iPSC) line CWRU191 (male).

### Example 2 Induction of OPCs and Oligodendrocytes

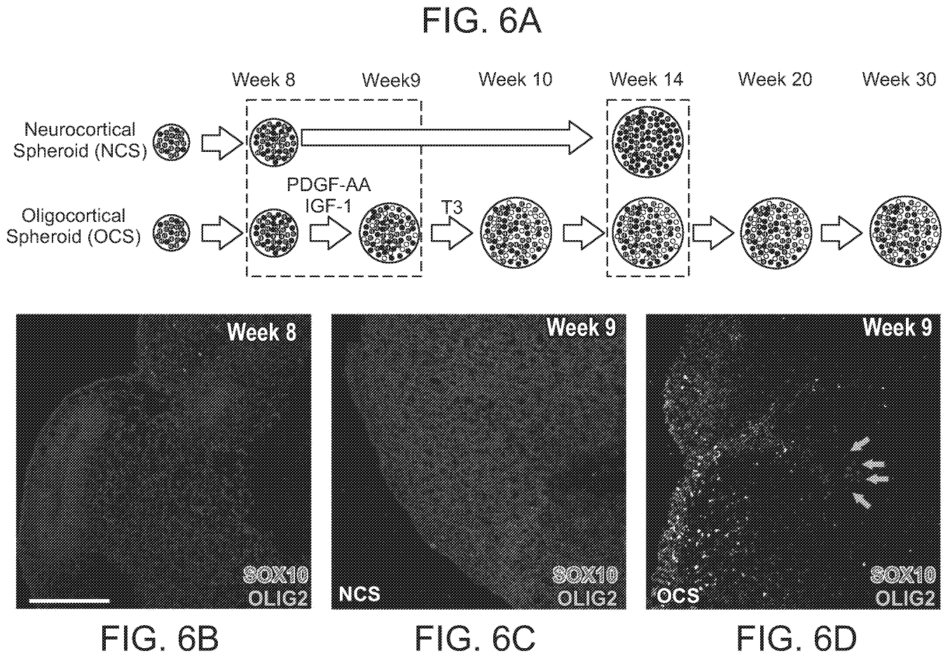

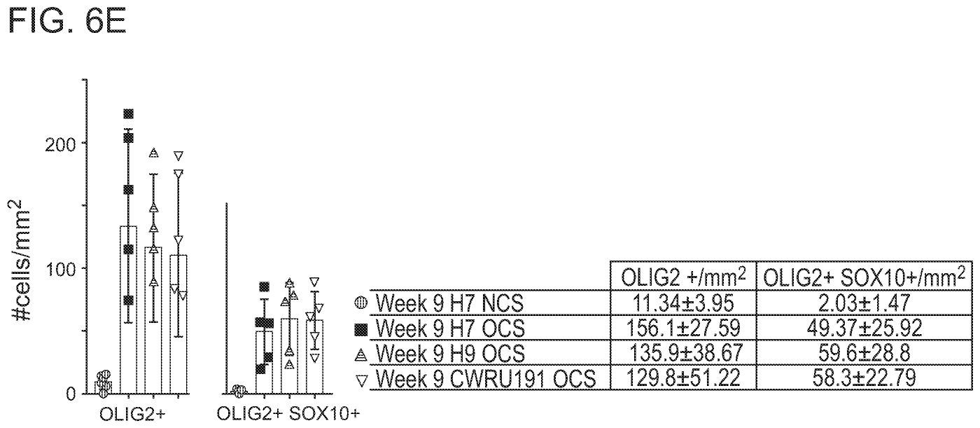

By the end of neurocortical patterning at week 8, neurocortical spheroids contained few cells in the oligodendrocyte lineage as evidenced by minimal immunostaining of OLIG2 and SOX10, two canonical OPC transcription factors (FIGS. 6B-6C). However, subsequent treatment of patterned spheroids with PDGF-AA and IGF-1 for 10 days, resulted in a substantial increase in the number of OPCs within the oligocortical spheroids compared to age-matched untreated neurocortical spheroids (FIGS. 6C-6E).

By week 14, neurocortical spheroids had generated robust populations of neurons and astrocytes, but no oligodendrocytes (FIG. 1B), while oligocortical spheroids (treated with PDGF-AA/IGF-1 from days 50-60 and T3 from days 60-70) reproducibly generated robust populations of oligodendrocytes across all three hPSC lines, as demonstrated by immunofluorescence for proteolipid protein 1 (PLP1), the most abundant oligodendrocyte membrane protein, and MYRF, a transcription factor specifically expressed in oligodendrocytes in the CNS (FIGS. 1C, 7A-7C).

Importantly, oligocortical spheroids exhibited low inter-line and inter-spheroid variability in the production of MYRF-positive oligodendrocytes: 21.59%±4.9%, 20.53%±3.9%, and 18.4%±2.2% of total cells (see FIG. 7C for quantification schematic) for H7, H9, and CWRU191 derived oligocortical spheroids, respectively, with n=5 spheroids per line (FIG. 1D).

Additionally, robust induction of the oligodendrocyte lineage was dependent on sequential treatment with both PDGF-AA/IGF-1 and T3, as few MYRF-positive oligodendrocytes were produced by either treatment individually (FIG. 1D).

Thus, while neurocortical patterning establishes the structural and cellular framework for oligodendrogenesis, PDGF-AA, IGF-1, and T3 are necessary for reproducible induction of OPCs and oligodendrocytes in this experiment.

To further validate the reproducibility of this approach, the protocol was replicated in an independent laboratory using an independent cell line, human embryonic stem cell line RUES1 (male), and separate personnel and reagents, wherein MYRF-positive cells constituted 18.36%±3.37% of cells in RUES derived oligocortical spheroids (FIGS. 1D, 7A-7B).

Lastly, RNA sequencing of bulk spheroids was used to globally assess how PDGF-AA/IGF-1 and T3 treatments affected transcription of neuron, astrocyte, and oligodendrocyte genes in oligocortical spheroids as compared to age-matched neurocortical spheroids. Analysis of week 14 spheroids for the expression of the 100 most specific mRNA transcripts for each cell type (defined using mouse transcriptional data from brainrnaseq.org) demonstrated no significant changes in neuronal gene sets, but showed a significant upregulation of glial gene sets, in particular those of the oligodendrocyte lineage (FIGS. 1E & 1F). These data demonstrate the method to generate oligocortical spheroids activates a global oligodendrocyte transcriptional program, but does not overtly alter the expression programs of other cell types in the spheroids, including neurons.

### Example 3 Oligodendrocyte Maturation and Myelination

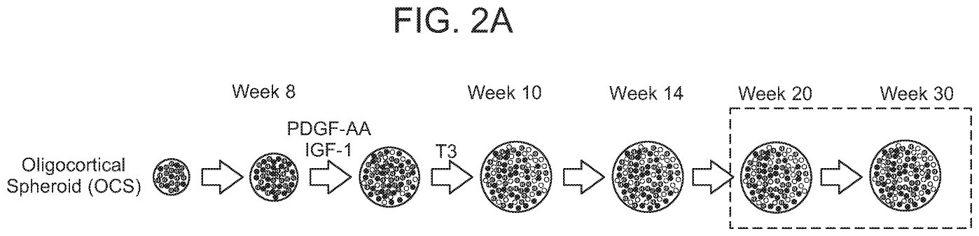

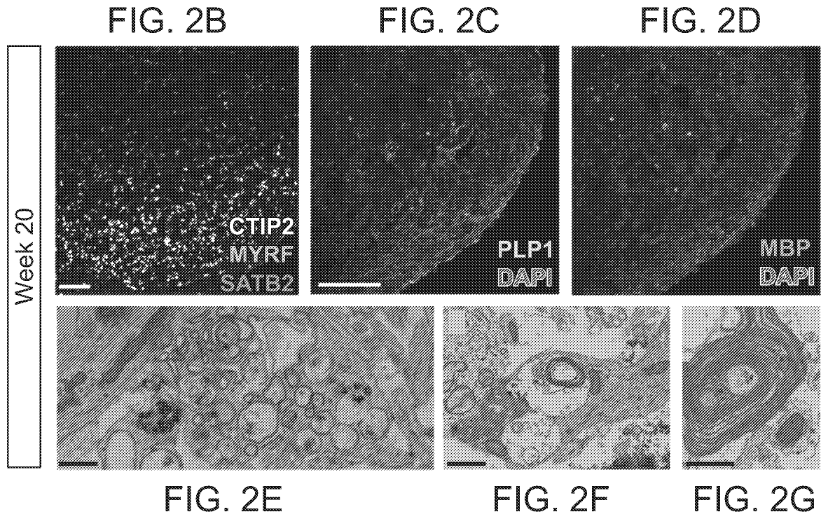

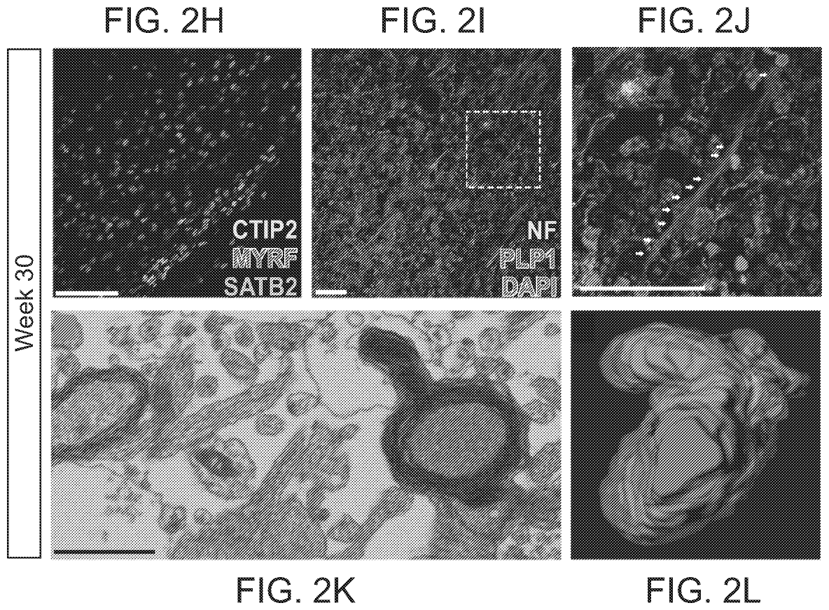

After initial oligocortical patterning, spheroids can be maintained in basal media for weeks to months. Applicant analyzed neuronal diversity and oligodendrocyte maturation at weeks 20 and 30 (FIG. 2A). Week 20 spheroids appear relatively immature. In addition to MYRF-positive oligodendrocytes, they contained a large population of early born deep layer neurons marked by CTIP2 and a separate smaller population of late born superficial layer neurons marked by SATB2, with MYRF-positive oligodendrocytes distributed throughout (FIGS. 2B, 8A). However, the neuron populations demonstrated substantial overlap, consistent with ongoing migration of younger SATB2 cells through the deep layers.

As oligodendrocytes mature, they extend cellular processes that track and myelinate adjacent axons. While PLP1 expression was robust as early as 14 weeks in culture, PLP1 immunofluorescence did not resolve into distinct processes until week 20 (FIGS. 2C, 8A). Furthermore, a subset of these processes began to express myelin basic protein (MBP, FIG. 2D), a marker of early myelin formation, suggesting that oligodendrocyte processes were associating with neuronal axons. Electron microscopy (EM) revealed concentric, but often unorganized, wrapping of human axons with multiple layers of uncompacted myelin (FIGS. 2E-2G, 8B) at week 20. While the unorganized nature of this early oligocortical spheroid myelin may be attributed, in part, to the in vitro culture environment, it does show striking resemblance to the earliest stages of in vivo fetal myelinogenesis in both human and chick. Importantly, despite T3 treatment and extensive oligodendrocyte maturation, week 20 oligocortical spheroids also maintained a pool of SOX10-positive, MYRF-negative OPCs (FIG. 8C).

At week 30, spheroids contained CTIP2- and SATB2-marked neuron populations organized into distinct cortical layers, with a large SATB2 population and a smaller CTIP2 layer. MYRF-positive oligodendrocytes were present both throughout these layers and as a distinct layer adjacent to CTIP2 (FIG. 2H). Additionally, oligodendrocyte processes had further resolved into distinct PLP1-positive tracts that co-localized with neurofilament-expressing neuronal axons (FIGS. 2I-2J). EM at week 30 identified neuronal axons encircled by compact myelin (FIG. 2K), and serial block face imaging with 3D reconstruction demonstrated longitudinal wraps of myelin ensheathing the axon (FIG. 2L). However, as of week 30, Applicant could not identify definitive evidence of further structural organization, such as nodes of Ranvier, likely due in part to the continued immaturity and minimal coherent electrical activity of spheroid neurons (a noted issue with all current spheroid and organoid technologies).

Collectively, these results demonstrate that early myelination of human neurons by human oligodendrocytes can be generated in the context of oligocortical spheroids in as little as 20 weeks, with myelin maturation, refinement, and compaction by 30 weeks. This in vitro timing is similar to the emergence of myelin in the latter part of the third trimester of human fetal development in utero, as well as the timing of human OPC maturation and myelination after transplantation to the rodent CNS, suggesting the potential presence of a cell-intrinsic developmental clock for human oligodendrocyte maturation as proposed in rodents.

### Example 4 Relevance to In Vivo Cortical Development

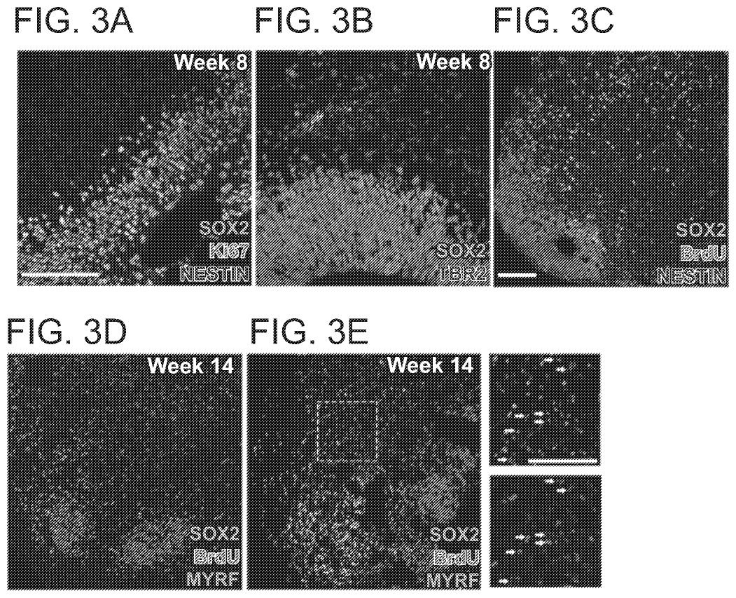

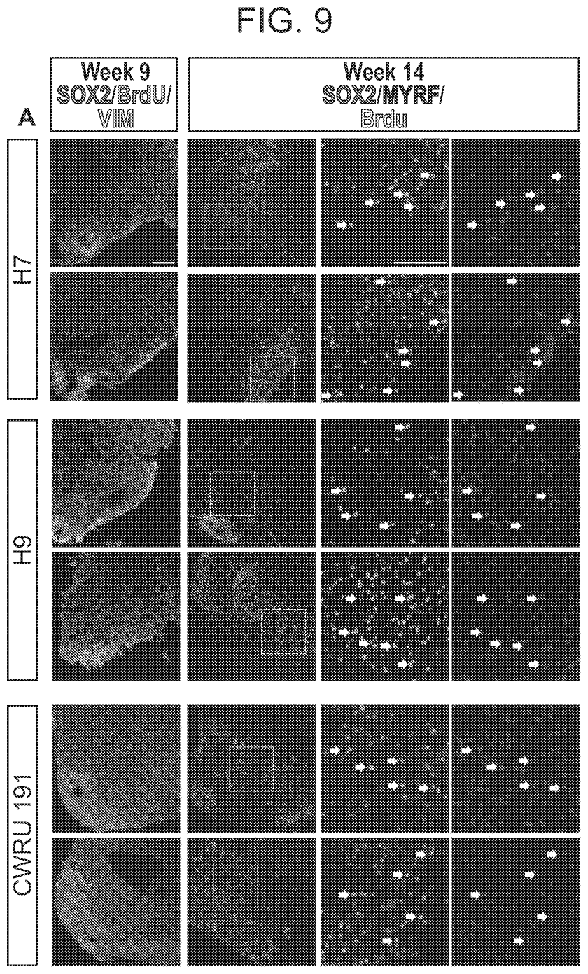

Applicant next evaluated the developmental and cellular organization within the subject oligocortical spheroids to demonstrate relevance to in vivo human cortical development. By week 8, spheroids contained robust populations of dividing Nestin-positive and SOX2-positive neural progenitors, organized into SOX2-positive ventricular-like and TBR2-positive outer subventricular-likes zones (FIGS. 3A and 3B). The arrangement of SOX2-positive germinal centers was reminiscent of the ventricular zone in the cortex, although not all SOX2 populations surrounded a ventricle-like void and many were localized to the outer surface of the spheroid. At week 9, Applicant labelled proliferating Sox2-positive cells of these germinal centers with the thymidine analog 5-bromo-2′-deoxyuridine (BrdU) (FIGS. 3C and 9A) and tracked their developmental trajectories. By week 14, BrdU-labelled cells had migrated away from the germinal center, forming a distinct population from the SOX2-positive germinal zones (FIGS. 3D-3E, 9A). At this time point, only oligocortical spheroids contained MYRF-positive OPCs, some of which were MYRF/BrdU-double positive (FIGS. 3E and 9A). MYRF co-localization with BrdU is strong evidence that these cells originated from BrdU labeled SOX2-positive progenitors found in progenitor zones of the oligocortical spheroids.

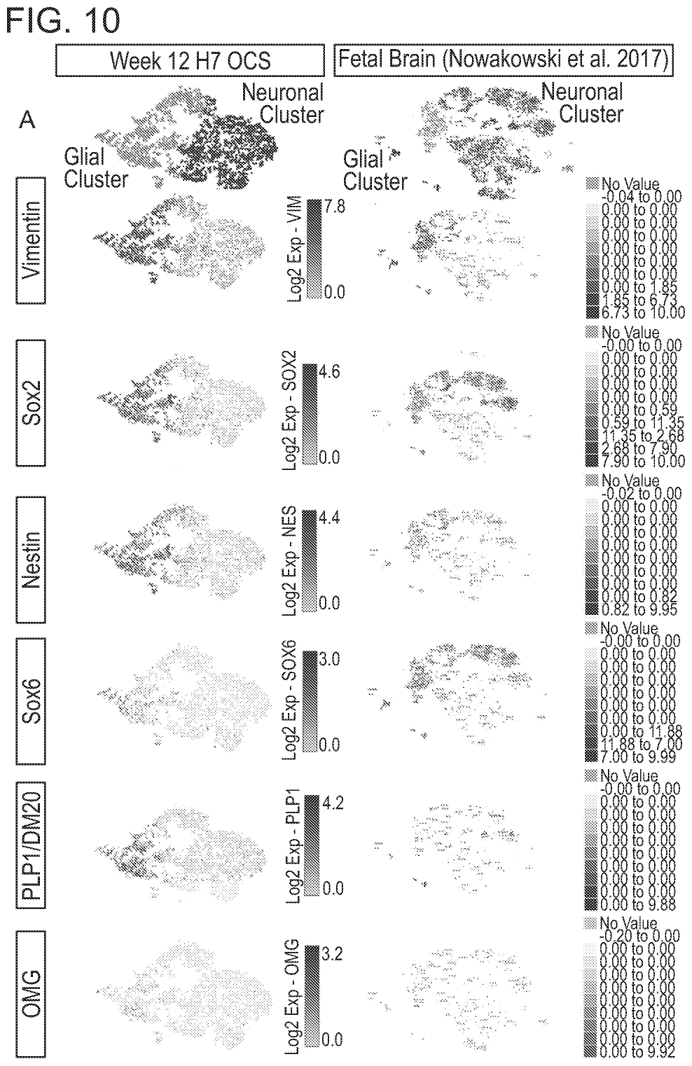

The migration of BrdU-pulsed progenitors away from germinal centers suggests that oligocortical spheroids contain a continuum of proliferative and differentiating oligodendrocytes. To assess the global diversity of cellular composition and spectrum of glial maturation, Applicant performed single-cell RNA-seq on week 12 oligocortical spheroids—an early time point just after PDGF-AA/IGF-1 and T3 treatment when all populations should be represented. Cell clustering broadly distinguished between glial and neuronal populations. The glial cluster contained early progenitors (marked by vimentin, SOX2, and nestin), OPCs (marked by SOX6), and maturing oligodendrocytes (marked by PLP1 and oligodendrocyte myelin glycoprotein) with expression of proliferative markers throughout the cluster and maturation markers defining progressively more distinct sub-populations (FIG. 10A). This single-cell analysis demonstrates that distinct populations of oligodendrocytes at multiple stages of development coexist in oligocortical spheroids, similar to single-cell transcriptome data from human fetal cortex (FIG. 10A). This suggests that oligocortical spheroids might provide an avenue to interrogate these largely inaccessible stages of human glial development.

### Example 5 Promyelinating Drug Tests in Spheroids

The ability to generate human oligodendrocytes that can myelinate human axons in an in vitro system provides new opportunities to explore human myelin development, disease, and therapeutics. Applicant first tested whether the subject human oligocortical spheroids recapitulate known effects of previously identified promyelinating drugs.

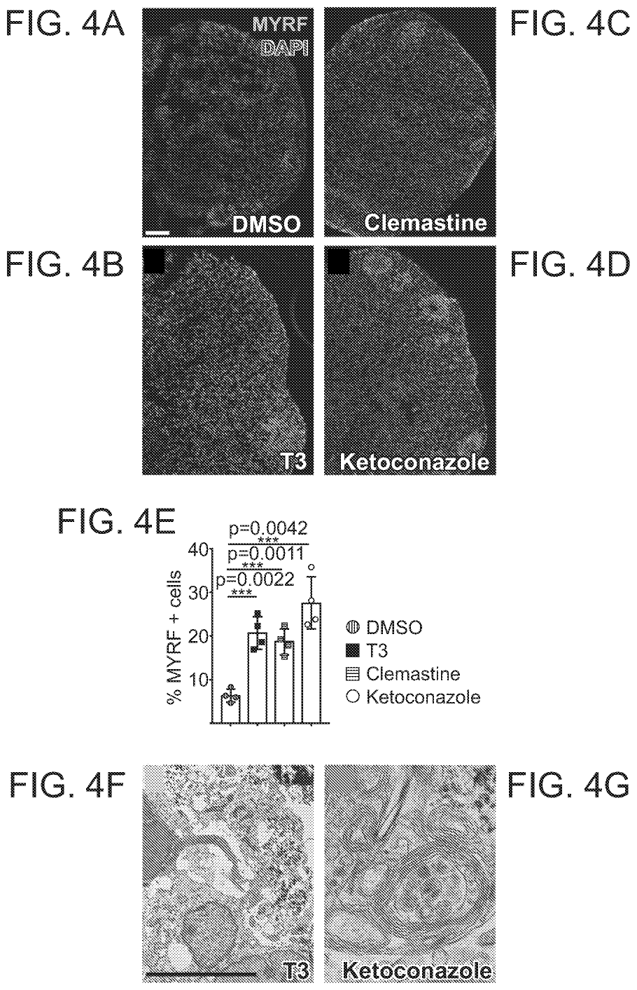

It has been shown that two FDA-approved drugs, clemastine and ketoconazole, are potent stimulators of rodent oligodendrocyte generation and myelination in vitro and in vivo. Moreover, clemastine was recently reported to enhance remyelination in a Phase 2 repurposing clinical trial in multiple sclerosis patients. To assess the effect of these promyelinating drugs on human oligodendrocyte generation, oligocortical spheroids were treated with PDGF-AA/IGF-1 from day 50-60, and then either DMSO, T3, clemastine, or ketoconazole from day 60-70, followed by a return to basal medium for 4 weeks. Quantification of MYRF-positive oligodendrocytes at week 14 revealed that clemastine (18.7%±2.94%) and ketoconazole (27.61%±5.941%) each enhanced the production of oligodendrocytes to a similar extent as T3 (21.59%±4.9%), compared to vehicle (DMSO) controls (6.345%±1.46%) (FIGS. 4A-4E). Remarkably, when examined by EM, ketoconazole-treated spheroids also exhibited myelination by week 14 of culture, two months earlier than T3 treated spheroids (FIGS. 4F-4G). These results demonstrate that clemastine and ketoconazole enhance and accelerate human oligodendrogenesis and maturation and validate that oligocortical spheroids provide a physiologic and species relevant preclinical model to evaluate candidate myelin therapeutics prior to human clinical trials.

### Example 6 Spheroids Recapitulate Pathology of a Myelin Disorder

Oligocortical spheroids provide an unprecedented tissue-like, minimally manipulated system in which to study hitherto inaccessible stages of human myelin formation and the pathologic processes leading to myelin disease. Applicant investigated the monogenic leukodystrophy Pelizaeus-Merzbacher disease (PMD [MIM 312080]) to test whether the subject system can recapitulate known cellular pathology and dysfunction.

PMD is a rare X-linked disease with defects in myelin production. Hundreds of mutations in the causal gene PLP1 have been identified in patients, who present with a spectrum of severity ranging from mild motor delay and spasticity to severe hypotonia with early childhood mortality.

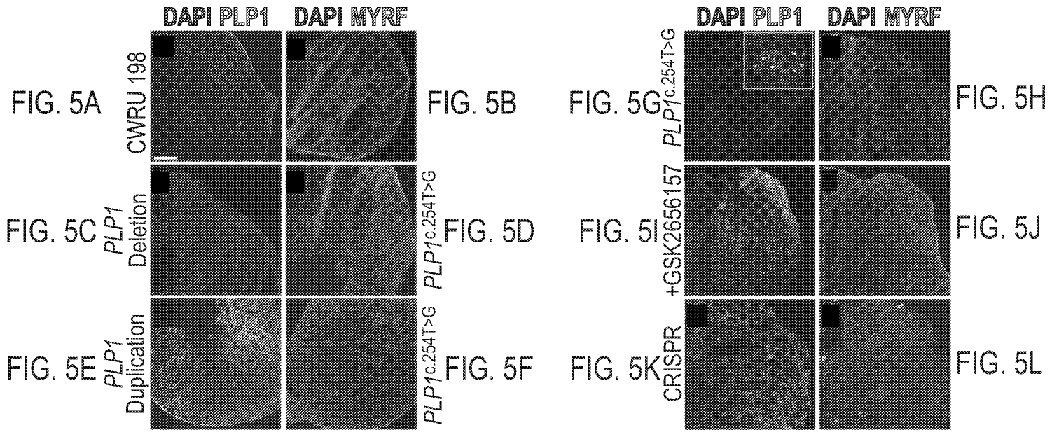

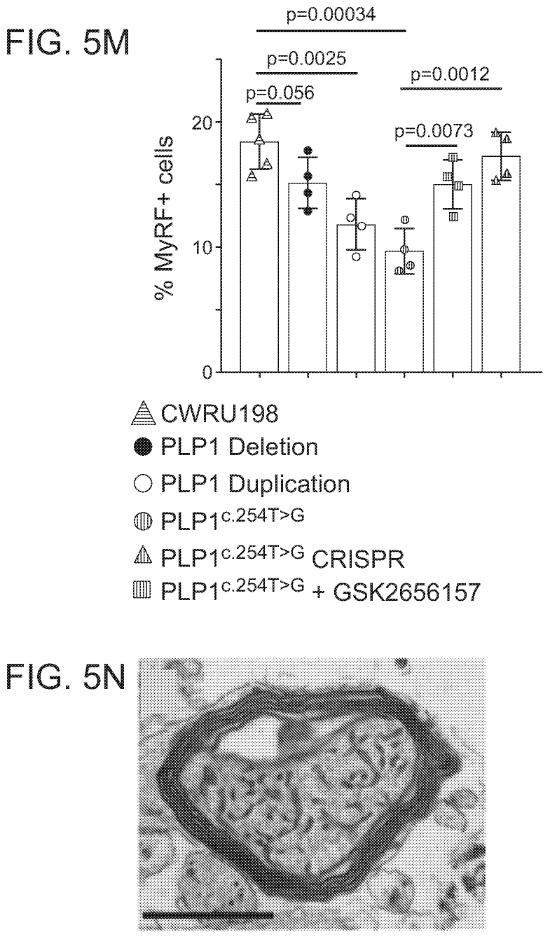

Applicant previously generated PMD iPSC-derived oligodendrocytes from a panel of affected male patients using two-dimensional (2D) culture and demonstrated both distinct and convergent cellular phenotypes in individuals with various mutations. Here, Applicant generated oligocortical spheroids from three iPSC lines with different PMD mutations: a deletion of the entire PLP1 locus, a duplication of the entire PLP1 locus, and a point mutation in PLP1 (c.254T>G). Phenotypically, these patients were mildly (deletion), moderately (duplication), and severely (point mutation) affected. To control for both gender and cell type of origin, Applicant simultaneously generated spheroids from a healthy control male iPSC line derived in-house, CWRU198, that expressed MYRF (18.4%±2.20%) and PLP1 (FIGS. 5A-5B) to similar extents as previously described control lines H7, H9, and CWRU191.

In oligocortical spheroids, the abundance of MYRF-positive oligodendrocytes trended with disease severity, while the extent of PLP1 expression correlated with genetic status (FIGS. 5C-5H). The PLP1 deletion line produced abundant MYRF-positive oligodendrocytes (15.14%±1.96%), despite the expected absence of PLP1 (FIGS. 5C-5D, 5M). Conversely, the duplication line produced abundant PLP1 signal (FIG. 5E), despite a significant decrease in MYRF-positive oligodendrocytes (11.84%±2.27%) compared to CWRU198 (FIGS. 5F, 5M).

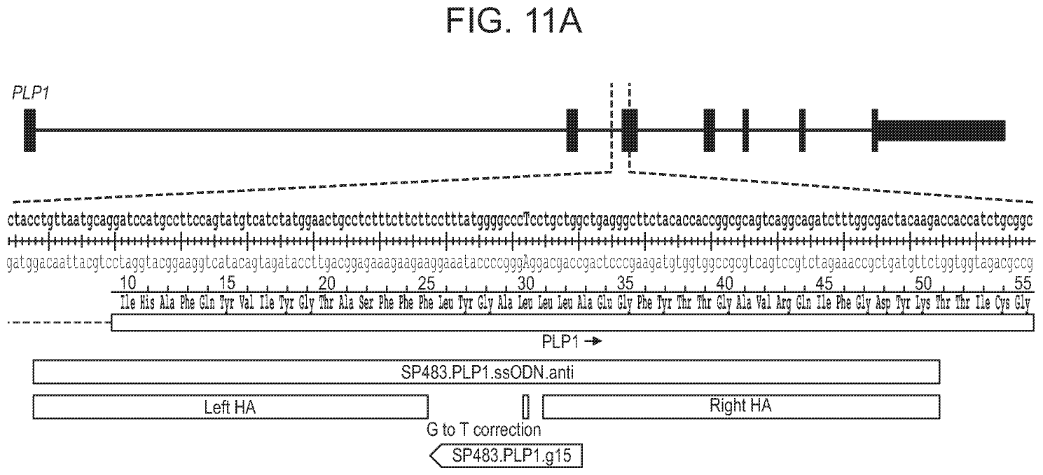

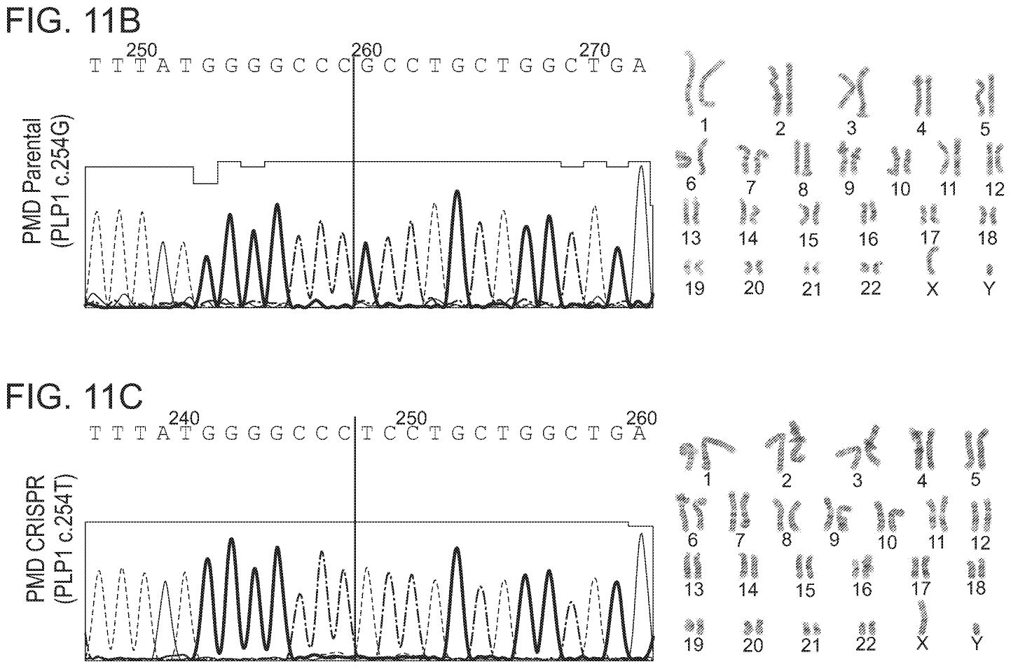

In previous 2D cultures, oligodendrocytes bearing the c.254T>G point mutation showed distinct perinuclear retention of PLP1, which resolved upon chemical modulation of the endoplasmic reticulum stress pathway. Oligocortical spheroids recapitulated this phenotype, demonstrating frank perinuclear retention of PLP1 (FIG. 5G) and the most severe reduction in MYRF-positive oligodendrocytes (9.69%±1.82%) (FIGS. 5H, 5M). Subsequent treatment of point mutation oligocortical spheroids with GSK2656157, an inhibitor of protein kinase R-like endoplasmic reticulum kinase (PERK), improved mobilization of PLP1 away from the endoplasmic reticulum and into oligodendrocyte processes (FIG. 5I) and significantly increased the percentage of MYRF-positive cells (15.04%±1.96%) (FIG. 5J, 5M). Lastly, CRISPR correction of the point mutation to the wild type sequence (FIGS. 11A-11C) in iPSCs prior to oligocortical spheroid generation not only restored PLP1 mobilization into oligodendrocyte processes (FIG. 5K), but also increased the percentage of MYRF-positive oligodendrocytes (17.25±3.22%) back to healthy control levels (FIG. 5L-5M) and enabled generation of myelin by 20 weeks in culture (FIG. 5N).

The mechanistic relationships between PMD genotypes and phenotypes have not been fully characterized. Current data suggest that accumulation of excess (e.g., duplicated) PLP1 or aberrant/misfolded (e.g., missense mutated) PLP1 leads to ER stress, cell death, and severe patient phenotypes, while PLP1 deletion is better tolerated, and the dichotomy between cell abundance and PLP1 expression in the subject oligocortical spheroids aligns with this hypothesis. hPSC-derived brain organoids and cortical spheroids have been used to dissect mutation-specific pathologic processes involved in neuronal disorders. Having validated the subject system, one can extend these efforts to a wide variety of myelin diseases and begin to explore patient-specific pathogenesis over the course of oligodendrocyte birth, maturation, myelination, and death.

### Example 7 Miscellaneous Methods

### Pluripotent Stem Cell Lines.

Healthy (CWRU191; CWRU198) and PMD iPSCs were generated previously after informed consent and approval of the Case Western Reserve University and University Hospital Institutional Review Board. Two human embryonic stem cell (hESC) lines from the approved NIH hESC Registry (“H7” NIHhESC-10-0061; “H9” NIHhESC-10-0062) were also used in these studies.

### Oligocortical Spheroid Differentiation

Neurocortical spheroids were generated from human pluripotent stem cells as previously described with variations noted below (Pasca et al., Functional cortical neurons and astrocytes from human pluripotent stem cells in 3D culture. Nat Methods 12, 671-678 (2015), incorporated herein by reference).

To pattern neurocortical spheroids, pluripotent stem cell colonies cultured on vitronectin (Gibco #A14700) were lifted using dispase (Gibco #17105-041) at 37° C. for 10 minutes. Intact colonies were transferred to individual low-adherence V-bottom 96-well plates (S-Bio Prime #MS-9096VZ) in 200 μL Spheroid Starter media with 10 μM Rock inhibitor Y-27632 (Calbiochem #688001), 10 μM Dorsopmorphin (Sigma #P5499), and 10 μM SB-431542 (Sigma #S4317). Spheroid Starter media was DMEM/F12 (Invitrogen #11320-033) containing 20% Knock out Serum (Invitrogen #12587-010), Non-essential amino acids (Invitrogen #11140050), Glutamax (Invitrogen #35050061), β-mercaptoethanol and 100 U/mL Penicillin/Streptomycin. The same media without rock inhibitor was used for the next five days, after which the media was changed to Neurobasal-A based spheroid media. Neurobasal-A spheroid media was Neurobasal-A medium (Invitrogen #10888022) with added B-27 serum substitute without vitamin A (Invitrogen #12587), Glutamax (Invitrogen #35050061) and 100 U/mL Penicillin/Streptomycin. From day 7-25, 20 ng/ml FGF-2 (R&D systems #233-FB-25/CF) and 10 ng/ml EGF (R&D systems #236-EG-200) were added to the media. Spheroids were cultured in 96-well plates through day 25, with daily half-media changes. On day 25, spheroids were transferred to ultra-low attachment 6-well plates (Corning #CLS3471) at a density of 8-10 spheroids per well and cultured thus through the remainder of the protocol. Also from this point forward 1% Geltrex (Invitrogen #A15696-01) was added to the Neurobasal-A spheroid media. Neural differentiation was induced between days 27 and 41 by supplementing Neurobasal-A spheroid media with 20 ng/ml BDNF (R&D systems #248-BD) and 20 ng/ml NT-3 (R&D systems #267-N). Half media changes were performed every other day between days 17 and 41.

To generate oligocortical spheroids, beginning on day 50, 10 ng/mL platelet-derived growth factor-AA (PDGF-AA, R&D Systems #221-AA-050) and 10 ng/mL insulin-like growth factor-1 (IGF-1, R&D Systems #291-G1-200) were added to the every-other-day media changes for 10 days. Next, on day 60, 40 ng/mL 3,3′,5-triiodothronine (T3, Sigma #ST2877) was added to the every-other-day media changes for 10 days. When used, small molecules were supplemented during this period. 4 μM Ketoconazole and 2 μM Clemastine were added in lieu of T3. GSK2656157 was added in addition to T3.

After day 70, spheroids were matured and maintained in Neurobasal-A spheroid media with every-other-day media changes until completion of the experiment.

### Independent Validation

One hESC line “RUES1” from the approved NIH hESC Registry (NIHhESC-09-0012) was used. RUES1 were cultured on matrigel in mTeSR1 medium (Stemcell Technologies #85850) and lifted using StemPro Accutase (Thermofisher #A1110501). Oligocortical spheroid differentiation was performed as described above, with the exception of using N2 supplement (Thermofisher #17502048) and 25 mg/mL human insulin solution (Sigma #I9278) in replacement of KSR for days 1-7 of the differentiation protocol.

### Small Molecules

4 mM stock solution of Ketoconazole (Sigma #K1003), 2 mM stock solution of Clemastine fumarate (Sigma #SML0445), and 10 mM stock solution of GSK2656157 (EMD Millipore #5046510001) were prepared, aliquoted, and stored at −20° C. Small molecules were warmed to 37° C. for 20 minutes before adding to pre-warmed medium. Frozen aliquots were thawed no more than twice before being discarded.

### BrdU Labeling

To label dividing cells in the spheroid, BrdU was added to culture media at a final concentration of 3 μg/mL on day 58 and day 60. Week 9 samples were collected 4 hours after BrdU administration on day 60. For lineage tracing experiments, BrdU labelled spheroids were collected on week 14 and processed for immunohistochemistry.

### PLP1 Gene Editing

CRISPR-Cas9 editing of a PLP1 point mutation (c.254T>G) in iPSCs was performed by the Genome Engineering and iPSC Center at Washington University in St. Louis using a guide RNA overlapping the mutation (sequence: CCAGCAGGCGGGCCCCATAAAGG) and a single strand oligonucleotide with 25 nucleotide homology arms surrounding the mutation. Upon receipt, the mutation and correction locus were resequenced and both lines were karyotyped to ensure no gross genotypic aberrations were generated during the editing process (Cell Line Genetics).

### Immunocytochemistry

Spheroids for immunohistochemistry were initially fixed with 4% ice-cold paraformaldehyde for 45 minutes, washed three times in PBS, and equilibrated with 30% sucrose overnight. The spheroids were embedded in OCT and sectioned at 10 μm.

Immunohistochemistry was performed as described previously (Najm et al., Nat Methods 8, 957-962 (2011)). Briefly, sections were washed in PBS three times and then blocked for 30 minutes in PBS containing 0.1% Triton X-100 and 0.25% Normal donkey serum. The sections were then incubated at 4° C. overnight using primary antibodies in blocking solution. Primary antibodies used: rat-anti-PLP1 (1:500, AA3, gift from Wendy Macklin); rabbit-anti-MYRF (1:1000, provided by Dr. Michael Wegner); goat anti-SOX10 (1:250 R&D Systems AF 2864); rabbit anti-OLIG2 (1:250, Millipore AB9610; mouse-anti-pan-axonal neurofilament (1:1000, Covance #SMI311); mouse-anti-MBP (1:200, Covance #Smi99), mouse-anti-pan-neuronal neurofilament (NF, 1:1000, Covance #SMI312); rabbit-anti-GFAP (1:1000, Dako #Z0334); mouse anti-SATB2 (1:250, Abcam, #ab51520); rat anti-CTIP2 (1:400, Abcam #ab18465); goat anti-SOX2 (1:250 R&D Systems, #AF2018); rabbit anti-TBR2 (1:250, Abcam, ab23345); mouse anti-Ki67 (1:250, Millipore MAB4190); mouse anti-Nestin (1:1000 Millipore, MAB5326); mouse anti-BrdU (1:1000, Millipore, MAB3510); chicken anti-Vimentin (1:1000 Abcam, ab24525); DAPI (1 μg/ml, Sigma #D8417).

Sections were then washed in PBS and incubated in secondary antibodies for 2 hours. All secondary antibodies were LifeTechnologies AlexaFluor conjugated secondary antibodies used at a dilution of 1:500.

In the case of PLP1 immunohistochemistry, a 20 minute wash in PBS containing 10% Triton X-100 was first performed prior to the blocking step. In the case of MBP immunohistochemistry, a 20 minute ice cold acetone post fixation step was used. BrdU immunohistochemistry was performed after antigen retrieval, which entailed placing slides in a sealed coplin jar with boiling 100 mM Sodium Citrate Buffer and allowing it to come to room temperature over the course of an hour.

Spheroid sections were imaged using either a Leica DMi8 fluorescence microscope or a Leica Sp8 confocal microscope at the Case Western Reserve School of Medicine Imaging Core. In order to count MYRF positive nuclei, four 20× fields were imaged per spheroid. Two fields from the top and bottom of the spheroid and 2 fields from the edges of the central region of the spheroids were quantified (see FIG. 6C for schematic). The total number of DAPI-positive cells and MYRF-positive cells were manually counted in Adobe Photoshop or NIH ImageJ. Three to five spheroids were analyzed per line and treatment condition and Graphpad Prism was used to perform a t-test to assess statistical significance between lines or treatments.

### Electron Microscopy

Spheroids were fixed and processed as previously described (Najm et al., Nat Methods 8, 957-962 (2011)). Samples were fixed for 1 hour at room temperature in a fixative solution containing 4% Paraformaldehyde (EMS), 2% Glutaraldehyde (EMS), and 0.1M Na Cacodylate (EMS). Samples were then osmicated, stained with uranyl acetate and embedded in EMbed 812 (EMS). Ultrathin sections (120 nm) from each spheroid sample were observed with a FEI Helios NANOLAB™ 660 FIBSEM using extreme high resolution (XHR) field emission scanning electron microscope equipped with a Concentric (insertable) higher energy electron detector, all images were taken using 4 Kv and 0.2 current landing voltage at high magnifications (15000-35000×).

### Serial Block Face Imaging and 3D-Reconstruction

Epoxy embedded spheroids were trimmed, mounted onto silicon wafers and covered by conductive silver paint. Using a sputter coating (Cressington Scientific Instruments) an additional iridium layer ˜1 nm was deposited and samples were loaded into a Helios Nanolab 660i dual beam microscope (FEI Company) for imaging. After setting up the ion column and beam coincidence at the eucentric height (tilt 52°), for electron beam 2 kV and 40 pA current landing was used, then ion beam (Ga⁺) assisted platinum was deposited as a protective layer for subsequent milling for cross section using low current 0.23 nA, while surplus block material was removed using a high ion beam current (30 kV, 6.5 nA).

For final surface polishing/milling, a reduced ion current was used (30 KV, 2.8 nA). For imaging, the Auto Slice and View G3 software (FEI Company) was used with an electron beam current of 400 pA, HFW 11.84 μm, to acquire an image stack of 154 sections (pixel size: 1.97, and z=50 nm) using TLD detector, with a resolution of 6144×4096, dwell time 6 μs, working distance 4.04 mm. Raw images were aligned in Fuji imaging processing package and Imaris 9.1 software (Bitplane AG) was used for image visualization and 3D-reconstruction of myelin bundle.

### Bulk RNA Sequencing and Analysis

Four spheroids per line were collected in TriReagent (Zymo Research #R2050-1-200) and RNA was extracted as per manufacturer's instructions. RNA was further purified using a Qiagen RNeasy Plus Mini kit (Qiagen, #73404). Illumina libraries were prepared and sequenced in 50 bp paired end mode on a HiSeq 2500 instrument at the CWRU Genomics Core facility. Reads were aligned to the hg19 genome using TopHat v2.0.6 without providing a reference transcriptome. Abundance of transcripts from the iGenomes hg19 RefSeq reference were measured using Cufflinks v2.0.2. FPKMs were quantile normalized. Neuron-, astrocyte- and oligodendrocyte-specific genes were defined by expression (FPKM>1) in their respective cells and absence in the other two lineages. Each list was reduced to the 100 genes most specific to that cell-type by fold-change that were also detected in at least one spheroid sample. Differences in expression of gene lists were assessed using the Wilcoxon test in Graphpad Prism.

### Single Cell RNA Sequencing and Analysis

Ten independently generated week 12 spheroids were pooled and dissociated as previously described (Marques et al., Science 352, 1326-1329 (2016)). Briefly, spheroids were dissociated using the Worthington Papain dissociation system (Worthington Biochemical Corp., Lakewood N.J., Cat #: LK003150) following the manufacturer's instructions. Papain solution was oxygenated with 95% O₂ and 5% CO₂ prior to dissociation. Cell counts of single cell suspension were performed on the Countess Automated Cell Counter (Invitrogen) and cells were loaded for single cell capture at a final concentration of 1,000 cells/μL.

Single cell capture, cDNA synthesis, cDNA preamplification, and library preparation were performed using the 10× Genomics Chromium Single Cell 3′ Library and Bead Kit v2 (10× Genomics Inc, Pleasanton Calif., Cat #: 120237). 3,850 cells were recovered and sequenced at a depth of 38,611 reads per cell with 1,870 median genes per cell. Cell Ranger Single-Cell Software Suite v2.1.0 was used for barcode processing and single-cell 3′ gene counting and reads were mapped to hg19. PCA dimensionality reduction and tSNE analysis was performed by Cell Ranger Single-Cell Software Suite v2.1.0 and data visualized using 10× Genomics Loupe Cell Browser v2.0.0. Data in FIGS. 2A-2L was clustered with 10× Genomics Loupe Cell Browser v2.0.0 using K-Means clustering with a present number of 2 clusters to isolate broad clusters of neuronal and glial/progenitor. Clustering of spheroids was compared to publically available single-cell data from developing human cortex and available on UCSC Cluster Browser (bit.ly/cortexSingleCell). Oligocortical spheroid gene expression cluster heatmaps in FIG. 2 were generated by 10× Genomics Loupe Cell Browser v2.0.0 and represent the Log 2Fold change of gene expression in each cell compared to the mean expression of that gene in the population as a whole. Comparative gene expression cluster heatmaps of developing human cortex were generated from the UCSC Cluster Browser.

### Life Sciences Reporting Summary

Further information on experimental design is available in the Life Sciences Reporting Summary.

### Data Availability

All RNA-seq data have been deposited to the Gene Expression Omnibus (GEO) database under the accession number GSE110006 (incorporated herein by reference).

### Statistics

In order to quantify the percentage of MYRF positive oligodendrocytes in a single spheroid, 4 regions (as shown in FIG. 7) were imaged and the percentage of MYRF cells were averaged per spheroid. For data shown in FIG. 1, five spheroids (n=5) were analyzed similarly per treatment group. For data shown in FIG. 4, four spheroids were analyzed in each group (n=4). Data presented in FIG. 5M, was obtained from five (n=5) spheroids of line CWRU198 and four spheroids from each PMD line (n=4). A two-tailed unpaired t-test with Welch's correction was performed to compare 2 groups at a time.

Bulk RNA-seq was performed using 5 spheroids from each condition. Paired non-parametric Wilcoxon matched pairs signed-rank test was used to determine statistical significance.

### REFERENCES

1. Kadoshima, T. et al. Self-organization of axial polarity, inside-out layer pattern, and species-specific progenitor dynamics in human ES cell-derived neocortex. Proc Natl Acad Sci USA 110, 20284-20289 (2013).
2. Lancaster, M. A. et al. Cerebral organoids model human brain development and microcephaly. Nature 501, 373-379 (2013).
3. Pasca, A. M. et al. Functional cortical neurons and astrocytes from human pluripotent stem cells in 3D culture. Nat Methods 12, 671-678 (2015).
4. Camp, J. G. et al. Human cerebral organoids recapitulate gene expression programs of fetal neocortex development. Proc Natl Acad Sci USA 112, 15672-15677 (2015).
5. Jo, J. et al. Midbrain-like Organoids from Human Pluripotent Stem Cells Contain Functional Dopaminergic and Neuromelanin-Producing Neurons. Cell Stem Cell 19, 248-257 (2016).
6. Bagley, J. A., Reumann, D., Bian, S., Levi-Strauss, J. & Knoblich, J. A. Fused cerebral organoids model interactions between brain regions. Nat Methods 14, 743-751 (2017).
7. Birey, F. et al. Assembly of functionally integrated human forebrain spheroids. Nature 545, 54-59 (2017).
8. Lancaster, M. A. et al. Guided self-organization and cortical plate formation in human brain organoids. Nat Biotechnol (2017).
9. Li, Y. et al. Induction of Expansion and Folding in Human Cerebral Organoids. Cell Stem Cell 20, 385-396 e383 (2017).
10. Quadrato, G. et al. Cell diversity and network dynamics in photosensitive human brain organoids. Nature 545, 48-53 (2017).
11. Renner, M. et al. Self-organized developmental patterning and differentiation in cerebral organoids. EMBO J (2017).
12. Sloan, S. A. et al. Human Astrocyte Maturation Captured in 3D Cerebral Cortical Spheroids Derived from Pluripotent Stem Cells. Neuron 95, 779-790 e776 (2017).
13. Xiang, Y. et al. Fusion of Regionally Specified hPSC-Derived Organoids Models Human Brain Development and Interneuron Migration. Cell Stem Cell 21, 383-398 e387 (2017).
14. Nakano, T. et al. Self-formation of optic cups and storable stratified neural retina from human ESCs. Cell Stem Cell 10, 771-785 (2012).
15. Pasca, S. P. The rise of three-dimensional human brain cultures. Nature 553, 437-445 (2018).
16. Arlotta, P. Organoids required! A new path to understanding human brain development and disease. Nat Methods 15, 27-29 (2018).
17. Lancaster, M. A. & Knoblich, J. A. Generation of cerebral organoids from human pluripotent stem cells. Nat Protoc 9, 2329-2340 (2014).
18. Luo, C. et al. Cerebral Organoids Recapitulate Epigenomic Signatures of the Human Fetal Brain. Cell Rep 17, 3369-3384 (2016).
19. Monzel, A. S. et al. Derivation of Human Midbrain-Specific Organoids from Neuroepithelial Stem Cells. Stem Cell Reports 8, 1144-1154 (2017).
20. McMorris, F. A., Smith, T. M., DeSalvo, S. & Furlanetto, R. W. Insulin-like growth factor I/somatomedin C: a potent inducer of oligodendrocyte development. Proc Natl Acad Sci USA 83, 822-826 (1986).
21. Noble, M., Murray, K., Stroobant, P., Waterfield, M. D. & Riddle, P. Platelet-derived growth factor promotes division and motility and inhibits premature differentiation of the oligodendrocyte/type-2 astrocyte progenitor cell. Nature 333, 560-562 (1988).
22. Barres, B. A., Lazar, M. A. & Raff, M. C. A novel role for thyroid hormone, glucocorticoids and retinoic acid in timing oligodendrocyte development. Development 120, 1097-1108 (1994).
23. Jakovcevski, I., Filipovic, R., Mo, Z., Rakic, S. & Zecevic, N. Oligodendrocyte development and the onset of myelination in the human fetal brain. Front Neuroanat 3, 5 (2009).
24. Silbereis, J. C., Pochareddy, S., Zhu, Y., Li, M. & Sestan, N. The Cellular and Molecular Landscapes of the Developing Human Central Nervous System. Neuron 89, 248-268 (2016).
25. Bujalka, H. et al. MYRF is a membrane-associated transcription factor that autoproteolytically cleaves to directly activate myelin genes. PLoS Biol 11, e1001625 (2013).
26. James, D., Noggle, S .A., Swigut, T. & Brivanlou, A. H. Contribution of human embryonic stem cells to mouse blastocysts. Dev Biol 295, 90-102 (2006).
27. Zhang, Y. et al. An RNA-sequencing transcriptome and splicing database of glia, neurons, and vascular cells of the cerebral cortex. J Neurosci 34, 11929-11947 (2014).
28. Nevin, Z. S. et al. Modeling the Mutational and Phenotypic Landscapes of Pelizaeus-Merzbacher Disease with Human iPSC-Derived Oligodendrocytes. Am J Hum Genet 100, 617-634 (2017).
29. Weidenheim, K. M., Kress, Y., Epshteyn, I., Rashbaum, W. K. & Lyman, W. D. Early myelination in the human fetal lumbosacral spinal cord: characterization by light and electron microscopy. J Neuropathol Exp Neurol 51, 142-149 (1992).
30. Szuchet, S., Nielsen, L. L., Domowicz, M. S., Austin, J. R., 2nd & Arvanitis, D. L. CNS myelin sheath is stochastically built by homotypic fusion of myelin membranes within the bounds of an oligodendrocyte process. J Struct Biol 190, 56-72 (2015).
31. Wang, S. et al. Human iPSC-derived oligodendrocyte progenitor cells can myelinate and rescue a mouse model of congenital hypomyelination. Cell Stem Cell 12, 252-264 (2013).
32. Windrem, M. S. et al. Human iPSC Glial Mouse Chimeras Reveal Glial Contributions to Schizophrenia. Cell Stem Cell 21, 195-208 e196 (2017).
33. Gao, F. B., Durand, B. & Raff, M. Oligodendrocyte precursor cells count time but not cell divisions before differentiation. Curr Biol 7, 152-155 (1997).
34. Raff, M. C., Lillien, L. E., Richardson, W. D., Burne, J. F. & Noble, M. D. Platelet-derived growth factor from astrocytes drives the clock that times oligodendrocyte development in culture. Nature 333, 562-565 (1988).
35. Temple, S. & Raff, M. C. Clonal analysis of oligodendrocyte development in culture: evidence for a developmental clock that counts cell divisions. Cell 44, 773-779 (1986).
36. Nowakowski, T. J. et al. Spatiotemporal gene expression trajectories reveal developmental hierarchies of the human cortex. Science 358, 1318-1323 (2017).
37. Najm, F. J. et al. Drug-based modulation of endogenous stem cells promotes functional remyelination in vivo. Nature 522, 216-220 (2015).
38. Mei, F. et al. Micropillar arrays as a high-throughput screening platform for therapeutics in multiple sclerosis. Nat Med 20, 954-960 (2014).
39. Cohen, J. A. & Tesar, P. J. Clemastine fumarate for promotion of optic nerve remyelination. Lancet 390, 2421-2422 (2017).
40. Green, A. J. et al. Clemastine fumarate as a remyelinating therapy for multiple sclerosis (ReBUILD): a randomised, controlled, double-blind, crossover trial. Lancet 390, 2481-2489 (2017).
41. Hobson, G. M. & Garbern, J. Y. Pelizaeus-Merzbacher disease, Pelizaeus-Merzbacher-like disease 1, and related hypomyelinating disorders. Semin Neurol 32, 62-67 (2012).
42. Douvaras, P. et al. Efficient generation of myelinating oligodendrocytes from primary progressive multiple sclerosis patients by induced pluripotent stem cells. Stem Cell Reports 3, 250-259 (2014).
43. Axten, J. M. et al. Discovery of GSK2656157: An Optimized PERK Inhibitor Selected for Preclinical Development. ACS Med Chem Lett 4, 964-968 (2013).
44. Garbern, J. Y. Pelizaeus-Merzbacher disease: Genetic and cellular pathogenesis. Cell Mol Life Sci 64, 50-65 (2007).
45. Bershteyn, M. et al. Human iPSC-Derived Cerebral Organoids Model Cellular Features of Lissencephaly and Reveal Prolonged Mitosis of Outer Radial Glia. Cell Stem Cell 20, 435-449 e434 (2017).
46. Mariani, J. et al. FOXG1-Dependent Dysregulation of GABA/Glutamate Neuron Differentiation in Autism Spectrum Disorders. Cell 162, 375-390 (2015).
47. Qian, X. et al. Brain-Region-Specific Organoids Using Mini-bioreactors for Modeling ZIKV Exposure. Cell 165, 1238-1254 (2016).
48. Pamies, D. et al. A human brain microphysiological system derived from induced pluripotent stem cells to study neurological diseases and toxicity. ALTEX 34, 362-376 (2017).
49. Kessaris, N. et al. Competing waves of oligodendrocytes in the forebrain and postnatal elimination of an embryonic lineage. Nat Neurosci 9, 173-179 (2006).
50. Miller, D. J. et al. Prolonged myelination in human neocortical evolution. Proc Natl Acad Sci USA 109, 16480-16485 (2012).
51. Sheng, Y. et al. Using iPSC-derived human DA neurons from opioid-dependent subjects to study dopamine dynamics. Brain Behav 6, e00491 (2016).
52. Najm, F. J. et al. Rapid and robust generation of functional oligodendrocyte progenitor cells from epiblast stem cells. Nat Methods 8, 957-962 (2011).
53. Trapnell, C., Pachter, L. & Salzberg, S. L. TopHat: discovering splice junctions with RNA-Seq. Bioinformatics 25, 1105-1111 (2009).
54. Trapnell, C. et al. Transcript assembly and quantification by RNA-Seq reveals unannotated transcripts and isoform switching during cell differentiation. Nat Biotechnol 28, 511-515 (2010).
55. Marques, S. et al. Oligodendrocyte heterogeneity in the mouse juvenile and adult central nervous system. Science 352, 1326-1329 (2016).

All references cited herein are incorporated herein by reference.

1. A method for generating an oligocortical spheroid (OCS) from pluripotent stem cells (PSCs), the method comprising:
a) generating a neurocortical spheroid (NCS) through neurocortical patterning of said pluripotent stem cells;
b) subjecting said neurocortical spheroid to timed exposure to defined oligodendrocyte lineage growth factors and/or hormones, to promote proliferation, survival and/or expansion of native oligodendrocyte progenitor cell (OPC) populations within said neurocortical spheroid, thereby generating the oligocortical spheroid;
wherein said oligocortical spheroid contain oligodendrocyte progenitor cells (OPCs) capable of differentiating into myelinating oligodendrocytes (ODCs) that are capable of myelinating axons.

2. The method of claim 1, wherein said defined oligodendrocyte lineage growth factors and hormones include platelet-derived growth factor-AA (PDGF-AA) and insulin-like growth factor-1 (IGF-1).

3. The method of claim 1 or 2, further comprising timed exposure to additional growth factors and/or hormones to induce oligodendrocyte differentiation.

4. The method of claim 3, wherein said additional growth factors and/or hormones comprise thyroid hormone (T3), clemastine, and/or ketoconazole.

5. The method of any one of claims 1-4, wherein step b) is carried out at a time equivalent to about 10 weeks post conception, or about 50-60 days after the beginning of step a).

6. The method of any one of claims 1-5, wherein said timed exposure to additional growth factors and/or hormones to induce oligodendrocyte differentiation is carried out at a time equivalent to about 14 weeks post conception, or about 60-70 days after the beginning of step a).

7. The method of any one of claims 1-6, wherein said pluripotent stem cells are from a human embryonic stem cell line, or from an induced pluripotent stem cell (iPSC) line.

8. The method of any one of claims 1-7, wherein step b) is carried out over a period of about 10 days.

9. The method of any one of claims 1-8, wherein said neurocortical spheroids at the end of step a) contain substantially no oligodendrocyte lineage cells (e.g., as evidenced by lack of or minimal immunostaining of one or more canonical OPC markers, such as transcription factors OLIG2 and SOX10).

10. The method of any one of claims 1-9, wherein said oligocortical spheroid at the end of step b) contains substantially increased OPCs compared to age-matched neurocortical spheroids untreated by step b) (e.g., as evidenced by increased immunostaining of one or more canonical OPC markers, such as a transcription factor such as OLIG2 and SOX10, an oligodendrocyte membrane protein such as proteolipid protein 1 (PLP1), and a transcription factor specifically expressed in oligodendrocytes in the CNS such as MYRF).

11. The method of any one of claims 1-9, wherein said pluripotent stem cells are iPSC isolated from a subject having a disease.

12. The method of claim 11, wherein said disease is characterized by a defect in myelin production, or a defect caused by/associated with loss of myelin or its function.

13. The method of claim 12, wherein said disease is Pelizaeus-Merzbacher disease (PMD).

14. The method of claim 12, wherein said PMD is characterized by a deletion of the entire PLP1 locus, a duplication of the entire PLP1 locus, or a point mutation in PLP1 (c.254T>G).

15. An oligocortical spheroid generated using the method of any one of claims 1-14.

16. An oligocortical spheroid developed from pluripotent stem cells, wherein said oligocortical spheroid contains oligodendrocyte progenitor cells (OPCs) capable of differentiating into myelinating oligodendrocytes that are capable of myelinating axons.

17. The oligocortical spheroid of claim 16, further comprising myelinating oligodendrocytes that are capable of myelinating axons.

18. A method for screening for a drug effective to treat a disease characterized by a defect in myelin production, the method comprising contacting a plurality of candidate drugs from a library of candidate drugs, each individually with an oligocortical spheroid developed from pluripotent stem cells from an individual having said disease, and identifying one or more candidate drugs that alleviate the defect in myelin production/restore myelin amount or function/prevent myelin loss as being effective to treat said disease.

19. The method of claim 18, further comprising administering the candidate drug identified as being effective to an animal having said disease.

20. The method of claim 18, wherein the individual is a human.

21. The method of claim 19, wherein said animal is a mouse as a model for said disease.
